(19) United States
(12) Patent Application Publication
(10) Pub. No.: US 2021/0115000 A1
(43) Pub. Date: Apr. 22, 2021

(54) NILE RED DERIVATIVES FOR IMPROVED RATIOMETRIC IMAGING FOR NERVE SPECIFIC CONTRAST

(71) Applicant: Oregon Health & Sciences University, Portland, OR (US)

(72) Inventors: Jesse R Korber, Portland, OR (US); Connor W Barth, Portland, OR (US); Summer L Gibbs, Portland, OR (US)

(21) Appl. No.: 17/050,370

(22) PCT Filed: Apr. 26, 2019
(86) PCT No.: PCT/US19/29378

§ 371 Date: Oct. 23, 2020

Related U.S. Application Data
(60) Provisional application No. 62/663,220, filed on Apr. 26, 2018.

(51) Int. Cl. C07D 265/38; A61K 49/00

(52) U.S. Cl. CPC C07D 265/38; A61K 49/0028

(57) ABSTRACT

Provided herein are novel Nile Red derivatives with varied electron donating and withdrawing groups to try to influence tissue-specific accumulation and generate a truly adipose specific fluorophore of Formula (I).

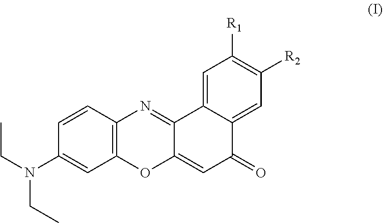

### ACKNOWLEDGEMENT OF GOVERNMENT SUPPORT

This invention was made with government support under R01 EB021362 and R01 CA188491 awarded by the National Institutes of Health. The government has certain rights in this invention.

### FIELD

The present invention is in the field of dyes for tissue specific contrast imaging. In particular, the present invention concerns novel adipose-specific dyes useful for fluorescence imaging and use in diagnosis and surgery.

### BACKGROUND OF THE INVENTION

Surgical nerve damage due to difficulty with identification remains a major risk for post-surgical complications and decreased quality of life. Fluorescence guided surgery offers a means to specifically highlight tissues of interest such as nerves and a number of fluorescence guided surgical systems are in clinical trial or approved for clinical use. However, no clinically approved nerve-specific fluorophores exist. Additionally, many pre-clinical nerve-specific fluorophores tend to accumulate in adipose tissue due to the molecular composition similarities between the two tissues, making it challenging to generate specific nerve signal. To alleviate this difficulty, we have synthesized a library of oxazine fluorophores based on the Nile Red scaffold, with the goal of strong adipose specificity without nerve uptake. The library was screened for tissue specificity ex vivo and in vivo, enabling quantification of adipose-, nerve- and muscle-specific uptake as well as selection of the best candidate for adipose selectivity without nerve signal. We showed our novel Nile Red fluorophore improved nerve to adipose and nerve to muscle contrast using ratiometric imaging as compared to the parent Nile Red compound. This adipose-specific Nile Red derivative could be used in future fluorescence guided surgery applications where adipose- or nerve-specific contrast were required.

Nerve damage sustained during surgery remains a major risk to patients, resulting in postsurgical complications such as pain, paralysis and persistent numbness.1, 2 Current techniques to avoid intraoperative nerve injury rely heavily on a surgeon's skill level and experience to visually avoid these typically small, buried structures. A contrast agent specific for nerve tissue could aid in identification and visualization of nerves during surgery, as a number of fluorescence image guided surgical systems are either in clinical trial or have FDA approval such as the FLARE™ by Curadel, Fluorobeam by Fluoptics, da Vinci robotic systems by Intutive Surical, SPY by Novadaq, Photodynamic Eye by Hamamatsu and Artemis by Quest Medical.3, 4 Imaging agents that can provide tissue-specific fluorescent signal have the potential to deliver a high contrast means of visualizing target tissues using such fluorescence guided surgical systems. However, currently there are only a handful of fluorescent contrast agents approved for clinical use including Fluorescein, Aminolevulinic Acid-Protoporphyin IX, Methylene Blue and Indocyanine Green, none of which have any nerve specificity (5-9)

A limited number of small molecule fluorescent contrast agents exist that stain nerve tissue in vivo, with six known classes of small molecule fluorophores that have been shown to have nerve or brain specificity. These include a handful of stilbene derivatives (10), a coumarin analog,11 a library of distyrylbenzene derivatives (12-15) 8 styryl pyridinium (FM) fluorophores (16,17) an oxazine fluorophore (2, 18) and a tricarbocyanine fluorophore (19). Unfortunately, adipopse specificity often accompanies nerve specificity for these small molecule fluorophores (2, 13, 14), which is largely by necessity since the molecular composition of nerve and adipose tissues are similar20, 21 making specific accumulation in the nerve without adipose accumulation a substantial challenge. This dual tissue accumulation compounds the problem of identifying and visualizing nerve tissue using fluorescence, as nerves are often adjacent to or embedded within adipose tissue. Of the known small molecule nerve-specific fluorophores, oxazines have demonstrated the greatest utility since the lead compound is red shifted and peripheral nerve specificity is high. However, the current lead nerve-specific oxazine compound, Oxazine 4, does not have the desired near infrared (NIR) excitation or emission, (2, 18) making additional chemical modifications necessary to generate a clinically relevant agent.

Interestingly, the commercially available Nile Red is also an oxazine fluorophore, however unlike Oxazine 4, it has a strong tendency to accumulate in lipid and adipose rich environments.22 Additionally, in an apolar environment such as lipids, Nile Red exhibits a high quantum yield and hypsochromatically shifted absorbance and emission spectra. In contrast, in a polar environment such as water, Nile Red exhibits bathochrommatically shifted absorbance and emission spectra with a quantum yield that approaches zero (23) These spectral properties have lead to a wide range of applications from evaluation of lipid droplets in tissue to its use in a PEGylated nanoparticle for iron detection (24) More recently, Nile Red has been shown to improve nerve-specific contrast of Oxazine 4 in a study that used dual fluorophore staining to enhance visualization of nerve tissue during in vivo studies where adipose, nerve and muscle tissues were present.2 While the adipose specificity of Nile Red was high, it showed some accumulation in nerve tissue after administration, reducing the contrast enhancing benefits of its use as a ratiometric fluorophore to enhance nerve contrast.

### SUMMARY OF THE INVENTION

Provided herein are novel Nile Red derivatives with varied electron donating and withdrawing groups to try to influence tissue-specific accumulation and generate a truly adipose specific fluorophore. Ex vivo and in vivo tissue staining studies were completed to assess the nerve, adipose and muscle accumulation as well as contrast from each Nile Red 4 derivative compared to the parent compound. Excitingly, a novel Nile Red derivative, Nile Red 5 (NR5) was found that showed adipose-specific contrast and decreased nerve accumulation as compared to Nile Red. Notably, NR5 also resulted in improved adipose to nerve and adipose to muscle contrast compared to Nile Red, especially when longer time points relevant to surgical interventions were considered. Ratiometic imaging with NR5 and Oxazine 4 compared to Nile Red and Oxazine 4 demonstrated improved nerve contrast using NR5 as compared to Nile Red.

These novel derivatives could be used to improve nerve-specific ratiometric imaging with Oxazine 4 or other nerve-specific fluorophores for fluorescence image guided surgery.

Provided are compounds of Formula (I):

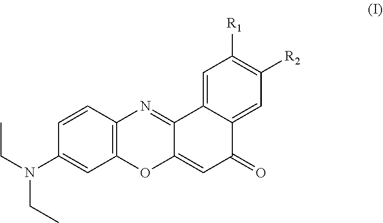

wherein:

one of R₁ and R₂ is hydrogen;

the other of R₁ and R₂ is selected from the group of:

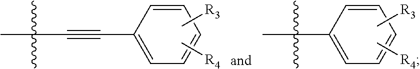

R₃ and R₄ are independently selected from H, —CN, —O—C₁-C₆ alkyl, —NH₂, NH(C₁-C₆ alkyl), —N(C₁-C₆ alkyl)₂, —CH═O, and —SO₂(C₁-C₆ alkyl);

with the proviso that not more than one of R₃ and R₄ is hydrogen.

### DESCRIPTION OF THE SEVERAL FIGURES

FIGS. 1A and 1B depict spectral properties of tested compounds.

FIG. 2A shows fluorescence from staining in ex vivo cross section of fixed mouse tissues.

FIG. 2B shows fluorescence from staining in ex vivo cross section of mouse tissues stained alive, then fixed.

FIG. 2C shows fluorescence assessed in live cells without a washing step.

FIG. 3A depicts ex vivo cross section of stained mouse tissues.

FIG. 3B contrasts nerve and adipose fluorescence staining from Nile Red and NR5 with lower muscle fluorescence staining intensity.

FIG. 3C represents documented adipose to nerve ratios obtained.

FIG. 4A provide in vivo imaging following direct administration of Nile Red and its derivatives localized to the adipose tissue.

FIG. 4B graphs fluorescent intensities of Nile Red and Nile Red derivatives in adipose, nerve, and muscle.

FIG. 4C compares generated adipose-to-muscle and adipose-to-nerve ratios.

FIG. 5A depicts in vivo mouse tissues 30 minutes post staining.

FIG. 5B compares adipose-specific fluorescence intensity with nerve and muscle tissue fluorescence.

FIG. 5C charts adipose-to-muscle and adipose-to-nerve ratios 30 minutes post direct administration.

FIG. 6A depicts ratiometric imaging after Nile Red or NR5 and Oxazine 4 co-staining

FIG. 6B represents area under the curve (AUC) and Z stat calculations from the ROC curve analysis for NR5 and Nile Red immediately following direct administration.

### DETAILED DESCRIPTION OF THE INVENTION

An embodiment herein provides compounds of Formula (I) wherein R₁ and R₂ are as defined above, and R₃ and R₄ are independently selected from H, —CN, —C₁-C₄ alkyl, —NH₂, NH(C₁-C₄ alkyl), —N(C₁-C₄ alkyl)₂, —CH═O, and —SO₂(C₁-C₄ alkyl); with the proviso that not more than one of R₃ and R₄ is hydrogen.

A further embodiment provides compounds of Formula (I) wherein R₁ and R₂ are as defined above, and R₃ and R₄ are independently selected from H, —CN, —C₁-C₃ alkyl, —NH₂, NH(C₁-C3₄ alkyl), —N(C₁-C₃ alkyl)₂, —CH═O, and —SO₂(C₁-C₃ alkyl); with the proviso that not more than one of R₃ and R₄ is hydrogen.

Another embodiment provides compounds of Formula (I) wherein R₁ and R₂ are as defined above, and R₃ and R₄ are independently selected from H, —CN, —O—CH₃, —NH₂, —NH—CH₃, —N(CH₃)₂, —CH═O, and —SO₂(CH₃); with the proviso that not more than one of R₃ and R₄ is hydrogen.

Still another embodiment provides compounds of Formula (I) wherein R₁ and R₂ are as defined above, and R₃ and R₄ are independently selected from H, —CN, —O—CH₃, —NH₂, —CH═O, and —SO₂(CH₃); with the proviso that not more than one of R₃ and R₄ is hydrogen.

Another embodiment provides compounds of Formula (I), above, wherein:

one of R₁ and R₂ is hydrogen;

the other of R₁ and R₂ is a group of the formula:

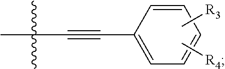

and

R₃ and R₄ are independently selected from H, —CN, —O—C₁-C₆ alkyl, —NH₂, NH(C₁-C₆ alkyl), —N(C₁-C₆ alkyl)₂, —CH═O, and —SO₂(C₁-C₆ alkyl); with the proviso that not more than one of R₃ and R₄ is hydrogen.

Another embodiment provides compounds of Formula (I), above, wherein:

one of R₁ and R₂ is hydrogen;

the other of R₁ and R₂ is a group of the formula:

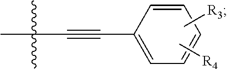

and

R₃ and R₄ are independently selected from H, —CN, —O—C₁-C₄ alkyl, —NH₂, NH(C₁-C₄ alkyl), —N(C₁-C₄ alkyl)₂, —CH═O, and —SO₂(C₁-C₄ alkyl); with the proviso that not more than one of R₃ and R₄ is hydrogen.

Another embodiment provides compounds of Formula (I), above, wherein:

one of R₁ and R₂ is hydrogen;

the other of R₁ and R₂ is a group of the formula:

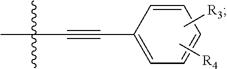

and

R₃ and R₄ are independently selected from H, —CN, —O—C₁-C₃ alkyl, —NH₂, NH(C₁-C₃ alkyl), —N(C₁-C₃alkyl)₂, —CH═O, and —SO₂(C₁-C₃ alkyl); with the proviso that not more than one of R₃ and R₄ is hydrogen.

Another embodiment provides compounds of Formula (I), above, wherein:

one of R₁ and R₂ is hydrogen;

the other of R₁ and R₂ is a group of the formula:

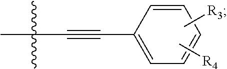

and

R₃ and R₄ are independently selected from H, —CN, —O—CH₃, —NH₂, —NH—CH₃, —N(CH₃)₂, —CH═O, and —SO₂(CH₃); with the proviso that not more than one of R₃ and R₄ is hydrogen.

Another embodiment provides compounds of Formula (I), above, wherein:

one of R₁ and R₂ is hydrogen;

the other of R₁ and R₂ is a group of the formula:

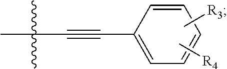

and

R₃ and R₄ are independently selected from H, —CN, —O—CH₃, —NH₂, —CH═O, and —SO₂(CH₃);

with the proviso that not more than one of R₃ and R₄ is hydrogen.

Another embodiment provides compounds of Formula (I), above, wherein:

one of R₁ and R₂ is hydrogen;

the other of R₁ and R₂ is a group of the formula:

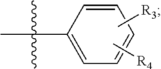

and

R₃ and R₄ are independently selected from H, —CN, —O—C₁-C₆ alkyl, —NH₂, NH(C₁-C₆ alkyl), —N(C₁-C₆ alkyl)₂, —CH═O, and —SO₂(C₁-C₆ alkyl); with the proviso that not more than one of R₃ and R₄ is hydrogen.

Another embodiment provides compounds of Formula (I), above, wherein:

one of R₁ and R₂ is hydrogen;

the other of R₁ and R₂ is a group of the formula:

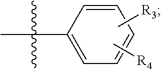

and

R₃ and R₄ are independently selected from H, —CN, —O—C₁-C₄ alkyl, —NH₂, NH(C₁-C₄ alkyl), —N(C₁-C₄ alkyl)₂, —CH═O, and —SO₂(C₁-C₄ alkyl); with the proviso that not more than one of R₃ and R₄ is hydrogen.

Another embodiment provides compounds of Formula (I), above, wherein:

one of R₁ and R₂ is hydrogen;

the other of R₁ and R₂ is a group of the formula:

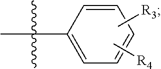

and

R₃ and R₄ are independently selected from H, —CN, —O—C₁-C₃ alkyl, —NH₂, NH(C₁-C₃ alkyl), —N(C₁-C₃alkyl)₂, —CH═O, and —SO₂(C₁-C₃ alkyl); with the proviso that not more than one of R₃ and R₄ is hydrogen.

Another embodiment provides compounds of Formula (I), above, wherein:

one of R₁ and R₂ is hydrogen;

the other of R₁ and R₂ is a group of the formula:

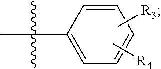

and

R₃ and R₄ are independently selected from H, —CN, —O—CH₃, —NH₂, —NH—CH₃, —N(CH₃)₂, —CH═O, and —SO₂(CH₃); with the proviso that not more than one of R₃ and R₄ is hydrogen.

Another embodiment provides compounds of Formula (I), above, wherein:

one of R₁ and R₂ is hydrogen;

the other of R₁ and R₂ is a group of the formula:

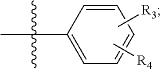

and

R₃ and R₄ are independently selected from H, —CN, —O—CH₃, —NH₂, —CH═O, and —SO₂(CH₃); with the proviso that not more than one of R₃ and R₄ is hydrogen.

Also provided are individual groups of compounds of Formula (II), Formula (III), Formula (IV), and Formula (V):

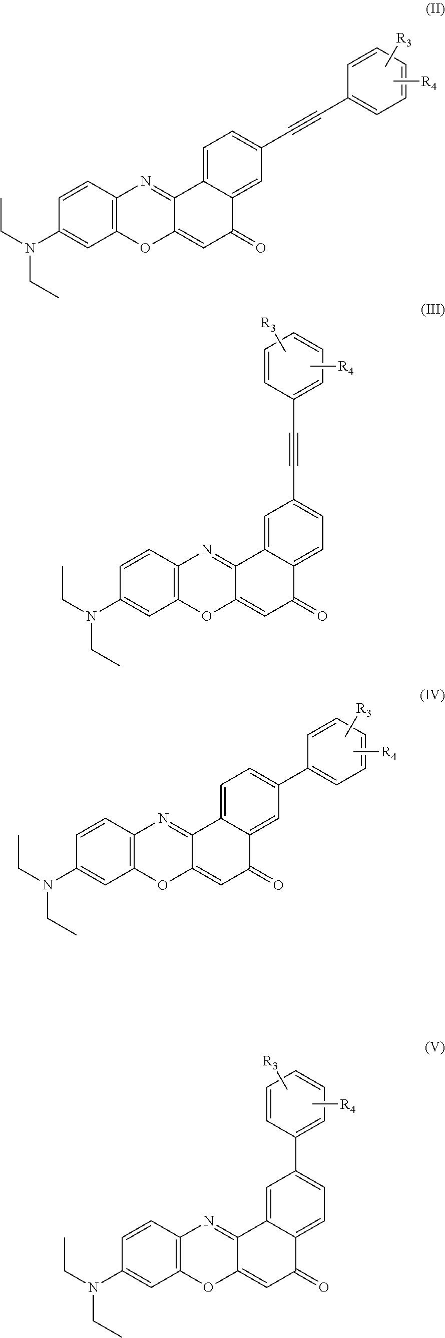

wherein, in each instance, R₃ and R₄ are independently selected from H, —CN, —O—C₁-C₆ alkyl, —NH₂, NH(C₁-C₆ alkyl), —N(C₁-C₆ alkyl)₂, —CH═O, and —SO₂(C₁-C₆ alkyl); with the proviso that not more than one of R₃ and R₄ is hydrogen.

Also provided are four separate groups of compounds of Formula (II), Formula (l), Formula (IV), and Formula (V), wherein, in each instance, R₃ and R₄ are independently selected from H, —CN, —O—C₁-C₄ alkyl, —NH₂, NH(C₁-C₄ alkyl), —N(C₁-C₄ alkyl)₂, —CH═O, and —SO₂(C₁-C₄ alkyl); with the proviso that not more than one of R₃ and R₄ is hydrogen.

Further provided are four separate groups of compounds of Formula (II), Formula (III), Formula (IV), and Formula (V), wherein, in each instance, R₃ and R₄ are independently selected from H, —CN, —O—C₁-C₃ alkyl, —NH₂, NH(C₁-C₃ alkyl), —N(C₁-C₃ alkyl)₂, —CH═, and —SO₂(C₁-C₃ alkyl); with the proviso that not more than one of R₃ and R₄ is hydrogen.

Additionally provided are four separate groups of compounds of Formula (II), Formula (III), Formula (IV), and Formula (V), wherein, in each instance, R₃ and R₄ are independently selected from H, —CN, —O—CH₃, —NH₂, —NH—CH₃, —N(CH₃)₂, —CH═, and —SO₂(CH₃); with the proviso that not more than one of R₃ and R₄ is hydrogen.

Provided also are four separate groups of compounds of Formula (II), Formula (III), Formula (IV), and Formula (V), wherein, in each instance, R₃ and R₄ are independently selected from H, —CN, —O—CH₃, —NH₂, —CH═O, and —SO₂(CH₃); with the proviso that not more than one of R₃ and R₄ is hydrogen.

Provided herein is the use of a compound of Formula (I) for fluorescence based imaging.

Also provided herein is an adipose-specific contrast composition comprising a compound of Formula (I) and a medically acceptable carrier, vehicle, solvent, emulsifier, or dispersing agent.

Further provided is a kit comprising a container with a composition comprising a medically useful amount of a compound of Formula (I) and a set of instructions for the use of the composition in an adipose imaging procedure.

Also provided is a kit comprising:
a) a first container containing a solution, emulsion, suspension, colloid, dispersion, or other mixture or form comprising a compound of Formula (I) in a concentration sufficient for fluorescence-based imaging;
b) a second container containing a solution, emulsion, suspension, colloid, dispersion, or other mixture or form comprising one or more nerve-specific fluorophores in sufficient concentration for fluorescence-based imaging; and
c) directions for the use of the elements a) and b), above, in fluorescence-based imaging.

In some embodiments, the nerve-specific fluorophore in the kits and methods herein may be selected from the group of distrylbenzenes (DSBs), oxazines (such as oxazine 4 perchlorate), and cyanines (such as 3,3′-diethylthiatricarbocyanine iodine).

Also provided herein is a method of contrasting adipose and nerve tissues or cells in an area of a subject's body or a sample therefrom, the method comprising the steps of:
a) staining the area of the subject's body or the sample therefrom with a contrast agent comprising an amount of a compound of Formula (I) sufficient for fluorescence imaging;
b) exciting the compound of Formula (I) staining the area of the subject's body or the sample therefrom with a light in the wavelength range capable of exciting the compound;
c) detecting the light emitted by the compound of Formula (I) staining the tissue or cell to create a first dataset;
d) staining the area of the subject's body or the sample therefrom with and amount of a nerve-specific contrast agent sufficient for fluorescence imaging;
e) exciting the nerve-specific contrast agent staining the area of the subject's body or the sample therefrom with a light in the wavelength range capable of exciting the compound; and
f) detecting the light emitted by nerve-specific contrast agent staining the tissue or cell to create a second dataset.

In some embodiments, the method of contrasting adipose and nerve tissues or cells above further comprises a step of generating images from the light emitted in steps c) and f), above.

In embodiments of the methods herein, the excitement of the compound or compounds of Formula (I) is completed by a light in the near infrared spectrum. In some embodiments, the preferred excitation/maximum absorption range for the compound of Formula (I) is from about 500 nm to about 900 nm. In other preferred embodiments, the excitation/maximum absorption range is between about 540 nm and about 600 nm. In other preferred embodiments, the excitation/maximum absorption range is between about 550 nm and about 680 nm. In further preferred embodiments, the excitation/maximum absorption range is between about 530 nm and about 565 nm. In additional preferred embodiments, the excitation/maximum absorption range is between about 550 nm and about 575 nm.

Also provided is the use of any compound disclosed herein in the preparation of a composition for use in imaging an adipose cell or tissue in a subject using near-infrared imaging.

It is understood that, for each reference to a compound of Formula (I) herein in regard to a composition, kit, method of use, method of preparation, etc., there are equivalent compositions, kits, methods of use, methods of preparation, etc., that utilize a compound of Formula (I) as further defined in each embodiments and descriptions herein. Also understood are equivalent compositions, kits, methods of use, methods of preparation, etc., that, respectively, utilize a compound of Formula (II), Formula (III), Formula (IV), Formula (V), and each of the individual contrast agents described herein.

### Definitions

The term “near infrared” or the acronym “(NIR)” refers to light at the near infrared spectrum, generally at a wavelength of about 0.65 to about 1.4 μm (700 nm-1400 nm. It may also refer to a range designated by the International Organization for Standardization as from a wavelength of about 0.78 μm to about 3 μm. In some embodiments, the preferred near infrared spectroscopy and imaging (NIRS) range is from about 500 nm to about 950 nm. In other embodiments, the preferred near infrared spectroscopy and imaging (NIRS) range is from about 500 nm to about 900 nm. In other preferred embodiments, the maximum absorption range is between about 540 nm and about 600 nm and the maximum emission range is from about 600 nm to about 650 nm. In other preferred embodiments, the maximum absorption range is between about 550 nm and about 680 nm and the maximum emission range is from about 620 nm to about 650 nm. In further preferred embodiments, the maximum absorption range is between about 530 nm and about 565 nm and the maximum emission range is from about 570 nm to about 640 nm. In additional preferred embodiments, the maximum absorption range is between about 550 nm and about 575 nm and the maximum emission range is from about 625 nm to about 650 nm. Among the commercially available light sources useful for excitation in the desired wavelengthsare the PhotoFluor® II and PhotoFluor II NIR devices available from 89 North, Williston, Vt.

The term “imaging” herein refers to the use of fluorescent compounds in conventional medical imaging techniques including, but not limited to, those related to fluorescence image-guided surgery (including minimally invasive laparoscopy or endoscopy techniques), computer-assisted surgery or surgical navigation, radiosurgery or radiation therapy, interventional radiology, fluorescence microscopy, and laser-confocal microscopy. These techniques may include near infrared wavelengths as described herein. In some embodiments the wavelength range is from about 650 nm to about 900 nm.

The term “nerve” means a bundle of neural axons. Within a nerve, each axon is surrounded by a layer of connective tissue called the endoneurium. The axons are bundled together into groups called fascicles, and each fascicle is wrapped in a layer of connective tissue called the perineurium. The entire nerve is wrapped in a layer of connective tissue called the epineurium. The term “nerve” is intended to include any tissues (e.g., the sinoatrial node or the atriventricular node) or structures associated therewith (e.g., neuromuscular junctions).

The term “adipose-specific” or “adipose specific” herein refers to an agent that is drawn to an adipose cell (adipocyte) or tissue and may be used in fluorescent imaging techniques to help contrast and differentiate the adipose cell or tissue from surrounding cells and/or tissues.

The term “medically acceptable” herein refers to a substance, such as a contrast agent vehicle, that may contact a cell, tissue, or organ without causing significant harm or negatively affecting the medical procedure or practice in which it is used, such as a fluorescence imaging technique.

The term “nerve-specific” or “nerve specific” herein refers to an agent that is drawn to a nerve or nerve tissue and may be used in fluorescent imaging techniques to help contrast and differentiate the nerve or nerve tissue from surrounding cells and/or tissues.

The term “subject” or “patient” refers to an animal, such as a mammal, that has been or will be the object of treatment, observation or experiment. The methods described herein may be useful in both human therapy and veterinary applications. In some embodiments, the subject is a mammal; in some embodiments the subject is human; and in some embodiments the subject is chosen from cats and dogs. “Subject in need thereof” or “human in need thereof” refers to a subject, such as a human, who may have or is suspected to have diseases or conditions that would benefit from certain treatment; for example a surgical or other intervention or treatment that may be enhanced by fluorescent imaging and a greater understanding of areas, tissues, or cells, particularly including the specific locations occupied by each.

The terms “surgery” or “surgical method” as used herein, refers to any method used to manipulate, change, or cause an effect by a physical intervention. These methods include, but are not limited to open surgery, endoscopic surgery, laparoscopic surgery, minimally invasive surgery, robotic surgery, any procedures that may affect any neuron or nerve, such as placement of retractors during spinal surgery, electrically conducting cardiac tissue or nerve ablation, epidural injection, intrathecal injections, neuron or nerve blocks, implantation of devices such as neuron or nerve stimulators and implantation of pumps. These methods may also include biopsy or other invasive techniques for the collection of cell or tissue samples, such as for diagnostic purposes.

Synthesis of Fluorophore Base Structures

### Chemicals and Solvents

All fluorophores, chemicals and solvents were purchased from Sigma Aldrich (St. Louis, Mo.) or Fisher Scientific (Hampton, N.H.) and used without further purification unless otherwise noted. All solvents were American Chemical Society (ACS) grade unless otherwise noted. Silica gel, 230-400 mesh was used for all column chromatography.

### Synthetic Confirmation & Fluorophore Characterization

Tandem liquid chromatography mass spectroscopy (LCMS) analysis (Agilent 1260 LC system with a 6224 time of flight (TOF) MS, Agilent, Santa Clara, Calif.) was performed using a 2.7 micron C18 column (4.6×50 mm, Agilent). High performance liquid chromatography (HPLC) purification (Agilent 1260 system with a preparative pump) was performed using a 5 micron C18 column (21.2×150 mm, Agilent). For both HPLC and LCMS, the mobile phase consisted of HPLC grade acetonitrile and water, treated with 0.1% formic acid. Spectroscopic analysis was performed on a Spectramax M5 spectrometer (Molecular Devices, Sunnyvale, Calif.) using a quartz cuvette with a 1 cm pathlength. Quantum yield was calculated according to equation (1).

$${∅}_{x} = {∅}_{s} \times \frac{{F}_{x}}{{F}_{s}} \times \frac{{f}_{s}}{{f}_{x}} \times \frac{{n}_{x}}{{n}_{s}} \qquad ( Eq . 1 )$$

Where Ø is the quantum yield, F is the integrated fluorescence intensity, f is the absorbance at the excitation wavelength and n is the refractive index of the solvent. S refers to the quantum yield standard (Rhodamine B)²⁵ and X refers to the unknown sample.

### Synthesis of Fluorophore Base Structures

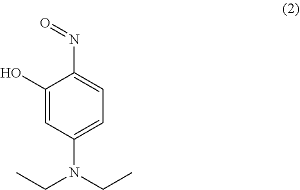

5-(diethylamino)-2-nitrosophenol (2): 1.02 g (6.16 mmol) of 3-diethylaminophenol (1) was dissolved in 16 mL of 2 M hydrochloric acid (HCl) and cooled in an ice bath for the duration of the reaction. 466.1 mg (6.76 mmol) of sodium nitrite (NaNO₂) was added dropwise, and the reaction was stirred for 2 h to yield the nitroso compound 2. The nitroso compound was filtered and used as the HCl salt without further purification (Scheme 1). ESI⁺ calculated for C₁₀H₁₅N₂O₂: 195.11 m/z; measured: 195.11 m/z.

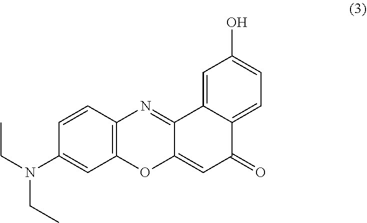 [SMILES: CCN(CC)c1ccc2nc3c4cc(O)ccc4c(=O)cc-3oc2c1]

9-(diethylamino)-2-hydroxy-5H-benzo[a]phenoxazin-5-one (3): 129.6 mg (0.81 mmol) of 1,6 dihydroxynapthalene and 142.9 mg (0.62 mmol) of 2 were dissolved in 3 mL of dimethylformamide (DMF) and heated to 100° C. under a blanket of nitrogen. The reaction was allowed to proceed for 4 hours monitored via thin layer chromatography (TLC) (dichloromethane (DCM):methanol (MeOH): 95:5) (Scheme 1). The solvent was removed under reduced pressure and the product was purified by column chromatography, eluting with chloroform (CHCl₃):Methanol (MeOH):Triethylamine (TEA), 100:3:2). ESI⁺ calculated for C₂₀H₁₉N₂O₃: 335.14 m/z; measured: 335.14 m/z.

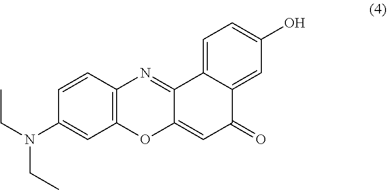 [SMILES: CCN(CC)c1ccc2nc3c4ccc(O)cc4c(=O)cc-3oc2c1]

9-(diethylamino)-3-hydroxy-5H-benzo[a]phenoxazin-5-one (4): 196.0 mg (1.22 mmol) of 1,7-dihyroxynaphthalene and 209 mg (0.91 mmol) of compound 2 were dissolved in 3 mL of DMF and heated to 100° C. under a blanket of nitrogen. The reaction was allowed to proceed for 4 hours monitored via TLC (DCM:MeOH, 95:5) (Scheme 1). The solvent was removed under reduced pressure and the product was purified by column chromatography, eluting with DCM:MeOH: (100:3). ESI⁺ calculated for C₂₀H₁₉N₂O₃: 335.14 m/z; measured: 335.14 m/z.

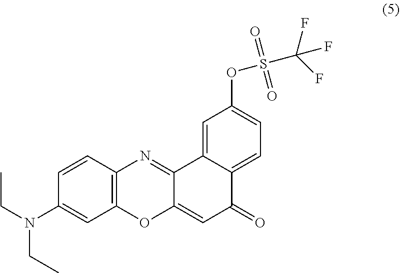 [SMILES: CCN(CC)c1ccc2nc3c4cc(OS(=O)(=O)C(F)(F)F)ccc4c(=O)cc-3oc2c1]

9-(diethylamino)-5-oxo-5H-benzo[a]phenoxazin-3-yl trifluoromethanesulfonate (5): 88.2 mg (0.25 mmol) of N-phenyl-bis(trifluoromethanesulfonimide) (Tf₂NPh) and 32 mg (0.10 mmol) of 3 were added to a scintillation vial and flushed with nitrogen. Anhydrous tetrahydrofuran (THF, 3 mL) followed by 400 μL (2.86 mmol) of TEA were added to the reaction vessel and it was tightly capped. The reaction was carried out overnight at room temperature. Solvent was removed under reduced pressure and the product was purified by column chromatography, eluting with CHCl₃:MeOH (100:3) (Scheme 1). ESI⁺ calculated for C₂₁H₁₈F₃N₂O₅S: 467.09 m/z; measured: 467.09 m/z.

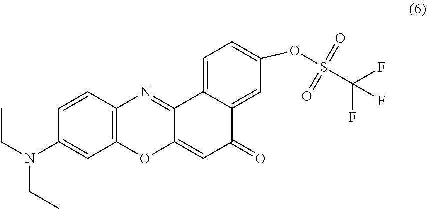 [SMILES: CCN(CC)c1ccc2nc3c4ccc(OS(=O)(=O)C(F)(F)F)cc4c(=O)cc-3oc2c1]

9-(diethylamino)-5-oxo-5H-benzo[a]phenoxazin-4-yl trifluoromethanesulfonate (6): 64.8 mg (0.181 mmol) of Tf₂NPh and 23.8 mg (0.0712 mmol) of 4 were added to a scintillation vial and flushed with nitrogen. Anhydrous THF (3 mL) and 284 μL (2.04 mmol) of TEA were added to the reaction vessel and it was tightly capped. The reaction was carried out overnight at room temperature. The solvent was removed under reduced pressure and the product was purified by column chromatography, eluting with CHCl₃:MeOH (100:3) (Scheme 1). ESI⁺ calculated for C₂₁H₁₈F₃N₂OS: 467.09 m/z; measured: 467.09 m/z.

### Sonogashira Nile Red Derivative Synthesis

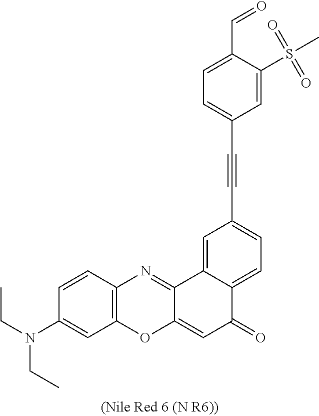 [SMILES: CCN(CC)c1ccc2nc3c4cc(C#Cc5ccc(C=O)c(S(C)(=O)=O)c5)ccc4c(=O)cc-3oc2c1]

4-((9-(diethylamino)-5-oxo-5H-benzo[a]phenoxazin-2-yl)ethynyl)-2-(methylsulfonyl)benzaldehyde (Nile Red 6 (NR6)): 1.6 mg (0.009 mmol) of palladium(II) chloride (PdCl₂), 1.8 mg (0.0095 mmol) of copper(I) iodide (CuI) and 2.3 mg (0.0088 mmol) of triphenylphosphine (PPh₃) were added to a round bottom flask. All solvents were sparged with nitrogen prior to use and the reaction was performed under a blanket of nitrogen. 6.1 mg (0.013 mmol) of 5 were dissolved in DMF (2 mL) and added to the flask followed by 53 μL (0.3803 mmol) of TEA. Trimethylsilylacetylene (TMS acetylene) was cooled in an ice bath and 53 μL (0.3826 mmol) were added to the reaction mixture. The reaction was run overnight at room temperature. The following morning 23.6 μL (0.156 mmol) of 1,8-diazabicyclo[5.4.0]undec-7-ene (DBU), 0.1 μL (0.006 mmol) of water and 11.4 mg (0.043 mmol) of 4-bromo-2-(methylsulfonyl)benzaldehyde were added to the reaction and it was heated to 70° C. for 24 hours (Scheme 2). The final compound was purified via HPLC with a water:acetonitrile gradient of 90:10 to 10:90 over 20 minutes. ESI⁺ calculated for C₃H₂₅N₂O₅S⁺: 525.15 m/z; measured: 525.15 m/z.

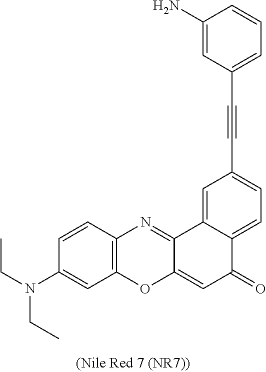 [SMILES: CCN(CC)c1ccc2nc3c4cc(C#Cc5cccc(N)c5)ccc4c(=O)cc-3oc2c1]

2-((3-aminophenyl)ethynyl)-9-(diethylamino)-5H-benzo[a]phenoxazin-5-one (Nile Red 7 (NR7)): 0.8 mg (0.0045 mmol) of PdCl₂, 1.9 mg (0.01 mmol) of CuI, and 2.4 mg (0.0092 mmol) of PPh₃ were added to a round bottom flask. All solvents were sparged with nitrogen prior to use and the reaction was performed under a blanket of nitrogen. 7.8 mg (0.0167 mmol) of 5 were dissolved in DMF (2 mL) and added to the flask followed by 53 μL (0.3803 mmol) of TEA. TMS acetylene was cooled in an ice bath and 53 μL (0.3826 mmol) were added to the reaction mixture. The reaction was run overnight at room temperature. The following morning 30 μL (0.20 mmol) of DBU, 0.120 μL (0.007 mmol) of water and 1.82 μL (0.167 mmol) of 3-bromoaniline were added to the reaction and it was heated to 70° C. for 3 hours (Scheme 2). The final compound was purified via HPLC with a water:acetonitrile gradient of 90:10 to 10:90 over 20 minutes. ES⁺ calculated for C₂₉H₂₃N₂O₃: 434.19 m/z; measured: 434.19 m/z.

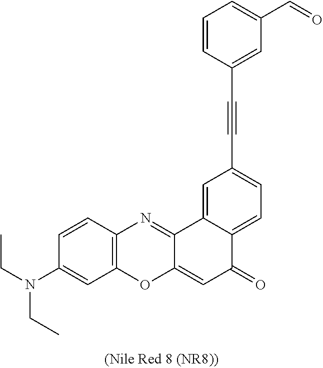 [SMILES: CCN(CC)c1ccc2nc3c4cc(C#Cc5cccc(C=O)c5)ccc4c(=O)cc-3oc2c1]

3-((9-(diethylamino)-5-oxo-5H-benzo[a]phenoxazin-2-yl)ethynyl)benzaldehyde (Nile Red 8 (NR8)): 1.0 mg (0.0056 mmol) of PdCl₂, 1.2 mg (0.0063 mmol) of CuI, and 2.7 mg (0.0103 mmol) of PPh₃ were added to a round bottom flask. All solvents were sparged with nitrogen prior to use and the reaction was performed under a blanket of nitrogen. 11.2 mg (0.024 mmol) of 5 were dissolved in DMF (2 mL) and added to the flask followed by 33 μL (0.2368 mmol) of TEA. TMS acetylene was cooled in an ice bath and 33 μL (0.2382 mmol) were added to the reaction mixture. The reaction was run overnight at room temperature. The following morning 43 μL (0.288 mmol) of DBU, 0.172 μL (0.0096 mmol) of water and 5.8 mg (0.025 mmol) of 3-iodobenzaldehyde were added to the reaction and it was heated to 75° C. for 3 hours (Scheme 2). The final compound was purified via HPLC with a water:acetonitrile gradient of 90:10 to 10:90 over 20 minutes. ES⁺ calculated for C₂₉H₂₃N₂O₃: 447.17 m/z; measured: 447.17 m/z.

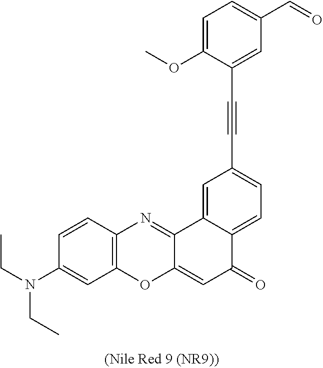 [SMILES: CCN(CC)c1ccc2nc3c4cc(C#Cc5cc(C=O)ccc5OC)ccc4c(=O)cc-3oc2c1]

3-((9-(diethylamino)-5-oxo-5H-benzo[a]phenoxazin-2-yl)ethynyl)-4-methoxybenzaldehyde (Nile Red 9 (NR9)): 1.4 mg (0.008 mmol) of PdCl₂, 1.7 mg (0.0089 mmol) of CuI, and 2.3 mg (0.0088 mmol) of PPh₃ were added to a round bottom flask. All solvents were sparged with nitrogen prior to use and the reaction was performed under a blanket of nitrogen. 13 mg (0.028 mmol) of 5 were dissolved in DMF (1 mL) and added to the flask followed by 53 μL (0.3803 mmol) of TEA. TMS acetylene was cooled in an ice bath and 53 μL (0.3826 mmol) were added to the reaction mixture. The reaction was run overnight at room temperature. The following morning 50.25 μL (0.336 mmol) of DBU, 0.2 μL (0.011 mmol) of water and 8.4 mg (0.039 mmol) of 4-bromo-2-(methylsulfonyl)benzaldehyde were added to the reaction and it was heated to 70° C. for 24 hours (Scheme 2). The final compound was purified via HPLC with a water:acetonitrile gradient of 90:10 to 10:90 over 20 minutes. ESI⁺ calculated for C₃H₂₅N₂O₄⁺: 477.18 m/z; measured: 477.18 m/z.

Suzuki Nile Red Derivative Synthesis

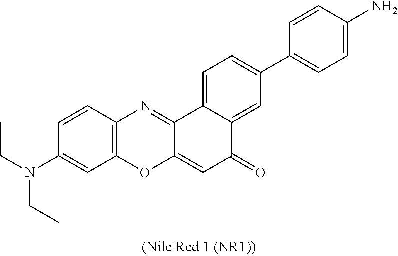 [SMILES: CCN(CC)c1ccc2nc3c4ccc(-c5ccc(N)cc5)cc4c(=O)cc-3oc2c1]

(3-(4-aminophenyl)-9-(diethylamino)-5H-benzo[a]phenoxazin-5-one (Nile Red 1 (NR1)): 14.5 mg (0.031 mmol) of 6, 0.3 mg (0.0013 mmol) of palladium (II) acetate (Pd(OAc)₂), 0.6 mg (0.0023 mmol) of PPh₃, 5.8 mg (0.1 mmol) of potassium fluoride (KF) and 7.8 mg (0.036 mmol) of 4-aminophenylboronic acid pincol ester were added to a round bottom flask and subsequently flushed with nitrogen. 1 mL of DMF was degassed under vacuum with sonication and added to the reaction vessel. The reaction was run at 70° C. overnight (Scheme 3). The final compound was purified via HPLC with a water:acetonitrile gradient of 90:10 to 10:90 over 20 minutes. ESI⁺ calculated for C₂₆H₂₄N₃O₂⁺: 410.19 m/z; measured: 410.19 m/z.

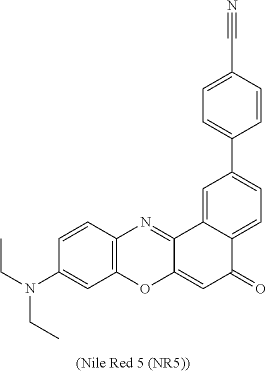 [SMILES: CCN(CC)c1ccc2nc3c4cc(-c5ccc(C#N)cc5)ccc4c(=O)cc-3oc2c1]

4-(9-(diethylamino)-5-oxo-5H-benzo[a]phenoxazin-3-yl)benzonitrile (Nile Red 5 (NR5)):

14.5 mg (0.031 mmol) of 6, 0.3 mg (0.0013 mmol) of Pd(OAc)₂, 0.6 mg (0.0023 mmol) of PPh₃, 5.8 mg (0.1 mmol) of KF and 6.4 mg (0.044 mmol) of 4-cyanophenylboronic acid were added to a round bottom flask and subsequently flushed with nitrogen. 1 mL of DMF was degassed under vacuum with sonication and added to the reaction vessel. The reaction was run at 70° C. overnight (Scheme 3). The final compound was purified via HPLC with a water:acetonitrile gradient of 90:10 to 10:90 over 20 minutes. ESI⁺ calculated for C₂₇H₂₂N₃O₂⁺: 420.17 m/z; measured: 420.17 m/z.

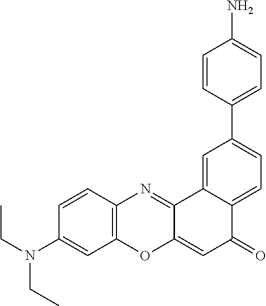 [SMILES: CCN(CC)c1ccc2nc3c4cc(-c5ccc(N)cc5)ccc4c(=O)cc-3oc2c1]

2-(4-aminophenyl)-9-(diethylamino)-5H-benzo[a]phenoxazin-5-one (Nile Red 10 (NR10)): 8.0 mg (0.017 mmol) of 5, 0.3 mg (0.0013 mmol) of Pd(OAc)₂, 0.5 mg (0.0019 mmol) of PPh₃, 3.1 mg (0.053 mmol) of KF and 5.0 mg (0.023 mmol) of 4-aminophenylboronic acid pinacol ester were added to a round bottom flask. The flask was flushed with nitrogen and nitrogen sparged THF (430 μL) was added. The reaction was run at 60° C. overnight (Scheme 3). The final compound was purified via HPLC with a water:acetonitrile gradient of 90:10 to 10:90 over 20 minutes. ESI⁺ calculated for C₂₆H₂₄N₃O₂⁺: 410.19 m/z; measured: 410.19 m/z.

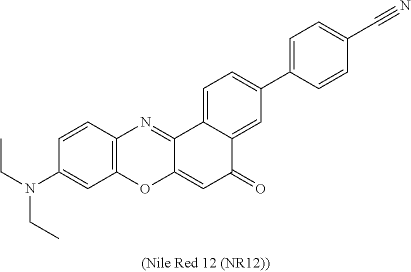 [SMILES: CCN(CC)c1ccc2nc3c4ccc(-c5ccc(C#N)cc5)cc4c(=O)cc-3oc2c1]

4-(9-(diethylamino)-5-oxo-5H-benzo[a]phenoxazin-2-yl)benzonitrile (Nile Red 12 (NR12)): 7.4 mg (0.022 mmol) of 5, 0.2 mg (0.0011) of PdCl₂, 0.7 mg (0.0027) of PPh₃ and 3.9 mg (0.0265 mmol) of 4-cyanophenylboronic acid were added to a round bottom flask. The flask was flushed with nitrogen and 200 μL of nitrogen sparged THF was added. A 2 M solution of potassium carbonate (K₂CO₃) was sparged with nitrogen and 64.5 μL was added to the reaction mixture. The reaction was run at 60° C. overnight (Scheme 3). The final compound was purified via HPLC with a water:acetonitrile gradient of 90:10 to 10:90 over 20 minutes. ESI⁺ calculated for C₂₇H₂₂N₃O₂: 420.17 m/z; measured: 420.17 m/z.

Cell Culture for Nile Red Derivative Staining

The U2OS human osteosarcoma cell line was cultured in Dulbecco's Modified Eagle Medium (DMEM, Thermo Fisher Scientific, Waltham, Mass.) with 10% fetal bovine serum (FBS, VWR, Radnor, Pa.) and 1% Penicillin/Steptomycin 100× (Thermo Fisher Scientific) at 37° C. in 5% CO₂. For live cell imaging studies, Fluorobrite DMEM media (Thermo Fisher Scientific) was used in place of DMEM. For imaging studies, cells were grown in 96-well glass bottom plates (Cellvis, Mountain View, Calif.) for 48 hours, with a final confluence of <80%. Nile Red and its derivatives were prepared at 1-10 mM stock concentration in DMSO and diluted into media or buffer for cell staining and imaging studies.

### Nile Red Derivative Cell Staining and Imaging Studies

Cell staining studies were completed on fixed and live U2OS cells using Nile Red and each synthesized Nile Red derivative described as follows. For fixed cell staining, cells were washed once with 1× phosphate buffered saline (PBS), then fixed with 4% paraformaldehyde (PFA) for 20 minutes at room temperature. The PFA was removed and each well was washed three times for 5 mins per wash (3×5 mins) with 1×PBS. The Nile Red fluorophores were diluted into 1×PBS to a final concentration of 1 μM, incubated for 5 minutes with the cells, and images were collected without washing. Live cell staining was completed using two methods. First, cells were washed once with Fluorobrite DMEM. The Nile Red fluorophore solution was prepared in Fluorobrite DMEM by diluting to a final concentration of 1 μM for staining, where cells were stained for 5 minutes. The fluorophore containing media was removed and the cells were fixed for 20 minutes using 4% PFA. Cells were then washed 3×5 mins with Fluorobrite DMEM prior to imaging. Second, cells were washed with Fluorobrite DMEM without FBS once for 5 minutes. Nile Red fluorophore solutions were prepared in Fluorobrite without FBS at 1 μM final concentration for cell staining. The cells were stained with the prepared solution for 5 minutes and imaged in the staining solution. All images were collected at their ideal exposure times to permit visualization of stained intracellular structures.

### Nile Red Derivative Ex Vivo Tissue Staining, Imaging and Fluorescence Quantification

Cryosections of CD-1 mouse sciatic nerve and surrounding muscle and adipose tissues from unrelated experiments were cut from optimal cutting temperature (OCT, Fisher Scientific, Hampton, N.H.) embedded frozen tissues. Cryosectioned tissues were captured on superfrost plus slides (Fisher Scientific) at 10 μm thickness. The tissue sections were washed with 1×PBS for 2 minutes to remove OCT, and then fixed with 2% PFA for 15 mins. The tissue sections were then washed with 0.1% Tween 20 in 1×PBS (3×5 mins), followed by incubation with 100 μM of Nile Red and each Nile Red derivative formulated using 10% DMSO, 5% Kolliphore (Sigma Aldrich, St. Louis, Mo.), 63.75% FBS, and 21.25% 1× PBS as previously described.²⁶ Tissue sections were incubated with the formulated fluorophores for 40 minutes at room temperature, followed by washing with blank formulation not containing fluorophore (2×5 mins). Each tissue section was then washed with 1×PBS (2×5 mins), fixed for 15 minutes using 2% PFA, followed by a final wash with 1×PBS for 5 mins before mounting in glycerol (Fisher Scientific). Images were collected at an exposure time of 650 ms for all Nile Red and Nile Red derivative stained sections. Four tissue sections were stained for Nile Red and each Nile Red derivative, facilitating adipose to muscle and adipose to nerve contrast ratio quantification where tissue intensities were quantified using region of interest analysis with custom written MatLab code (The MathWorks, Natick, Mass.). For each tissue type, a representative area was selected, where a threshold based mask was set to determine tissue from background. The mean tissue intensity was calculated in adipose, nerve and muscle tissues as the intensity per second and used to calculate the adipose to nerve and adipose to muscle contrast ratios.

### Fluorescence Microscopy

All ex vivo and cellular fluorescence and white light images were collected using a Zeiss AxioObserver inverted fluorescence microscope (Carl Zeiss, Oberkochen, Germany). All cell images were collected at 40× magnification and all tissue section images were collected at 10× magnification. Fluorescence excitation was completed using a Photofluor II broad band light source (89 North, Burlington, Vt.) filtered with a 545±12.5 nm bandpass excitation filter. Fluorescence images were collected using a 605±35 nm bandpass emission filter and a AxioCam 506 monochromo camera (Zeiss). All Filters were Purchased from Chroma Technologies (Bellows Falls, Vt.).

### In Vivo Imaging Following Direct/Topical Administration in Rodent Nerves

In vivo images were acquired using a previously published, custom-built small animal fluorescence imaging system, described briefly here.²⁷ Fluorescent images were obtained using a Qimaging EXi Blue monochrome camera (Surrey, British Columbia, CA) with a removable Bayer mask for collection of co-registered color and fluorescence images. A Photofluor II broad band light source (89 North) was expanded onto the surgical field through a liquid light guide and used unfiltered for white light illumination. Nile Red and NR5 were excitated using a 545 t 12.5 nm bandpass filter, while fluorescence emission images were collected with a 605±35 nm bandpass filter. Oxazine 4 (Exciton, West Chester, Ohio) was used for nerve and adipose co-staining and ratiometic imaging studies. Oxazine 4 was excited using a 620±30 nm bandpass filter, while fluorescence emission images were collected through a 700±37.5 nm bandpass filter.

Approval for the use of animals in the study was obtained from the Institutional Animal Care and Use Committee (IACUC) at Oregon Health and Science University (OHSU). In vivo staining studies were carried out on male CD-1 mice using an optimized direct/topical administration protocol.² Staining was performed concurrently on no more than two nerve sites (sciatic nerve or brachial plexus) per mouse. Nile Red and all Nile Red derivatives were formulated in a previously published co-solvent formulation (10% DMSO, 5% Kolliphor, 63.75% serum and 21.25% PBS¹⁴) at a final concentration of 100 μM. Each formulated fluorophore was incubated for 5 minutes on the nerve site (n=4 nerve sites/fluorophore), followed by 9 short flushes with PBS. The nerve site was then incubated with blank co-solvent not containing fluorophore for 5 minutes, followed again by 9 short flushes with PBS.

In vivo co-staining was completed with (1) Nile Red and Oxazine 4 as well as (2) NR5 and Oxazine 4. For these studies, co-solvent formulated Oxazine 4 (125 M) was incubated on the nerve site for 5 minutes followed by 9 short flushes with PBS. The co-solvent formulated Nile Red or NR5 (100 μM) was then incubated on the nerve site for 5 minutes and also followed by 9 short flushes with PBS. Co-registered (1) Nile Red and Oxazine 4 or (2) NR5 and Oxazine 4 images were collected from n=4 nerve sites per groups (1) and (2). Ratiometric images were calculated and quantified using region of interest analysis on the white light images to identify the nerve, adipose and muscle tissue types. Due to the known cross talk of Oxazine 4 into the Nile Red channel,² the Oxazine 4 image was subtracted from the Nile Red or NR5 image resulting in an image of only adipose. The ratiometric images were then calculated as the Oxazine 4 image minus the corrected Nile Red or NR5 image divided by the corrected Nile Red or NR5 image. Receiver Operator Characteristic (ROC) curve analysis was performed to assess differences between the ratiometric images, where the area under the curve (AUC) was used as the metric to differentiate between groups (1) and (2) using custom written MatLab code.²⁸

### Results

### Synthesis of the Nile Red Core Derivatives

The syntheses of the Nile Red core structures were completed in accordance with previously described methods (Scheme 1).²⁹ The nitroso compound 2 was prepared by reacting 3-diethylaminophenol with sodium nitrite on ice in HCl for two hours. The product was filtered and used immediately without further purification. Syntheses of compounds 3 and 4 were completed by condensation of 2 and either 1,6-dihydroxynapthalene or 1,7-dihydroxynapthalene at 100° C. The resulting 2-hydroxy or 3-hydroxy Nile Red derivatives were converted to their triflated versions (compounds 5 and 6) via reaction with n-phenyl bis(trifluoromethanesulfonimide) at room temperature. Compounds 5 and 6 were readily purified using column chromatography (CH₃Cl:MeOH, 100:3).

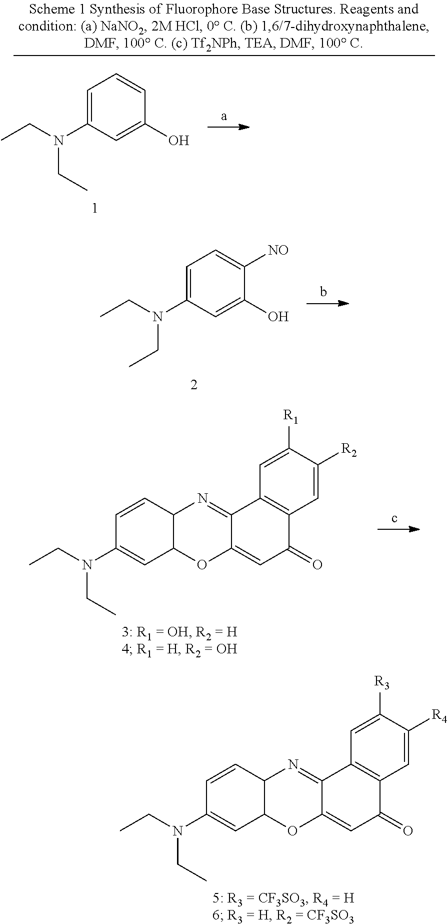

Nile Red derivatives NR6, NR7, NR8 and NR9 were prepared using a one pot Sonogashira reaction (Scheme 2).³⁰ Sparging with nitrogen was sufficient to removed dissolved oxygen from the solvent, and the reactions were carried out overnight with a catalytic amount of palladium, PPh₃ and CuI, in the presence of excess TEA. To induce the addition of the target aryl group, the aryl halide was added with catalytic water and excess DBU. The reaction was heated to 70° C. and run for 3-24 h.

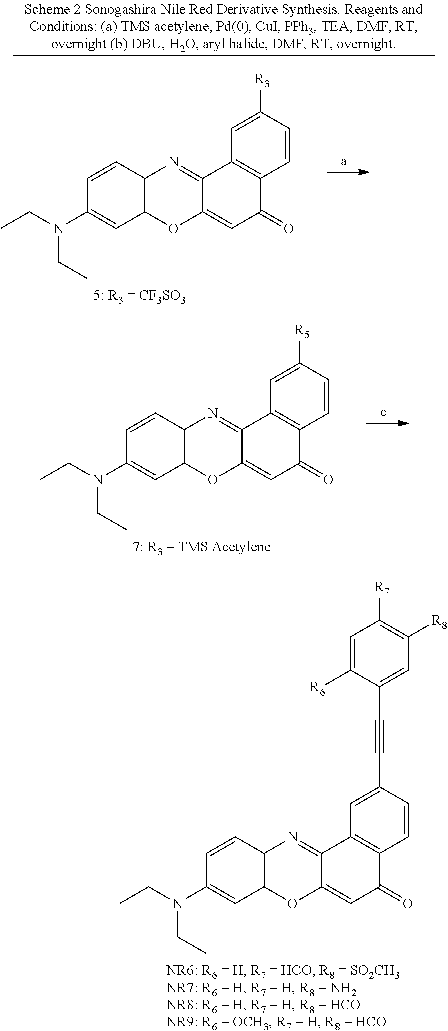

Nile Red derivatives NR1, NR5, NR10, and NR12 were prepared using a Suzuki coupling reaction (Scheme 3).^{31, 32} Rigorous degassing with sonication and house vacuum prior to use of any solvents yielded the most reliable reactions, as reagents were oxygen sensitive. Boronic acids, KF, the triflated Nile Red compounds, a catalytic amount of Pd(OAc)₂ and PPh₃ were added to the reaction vessel under nitrogen. The reactions was heated to 60-70° C. and run overnight.

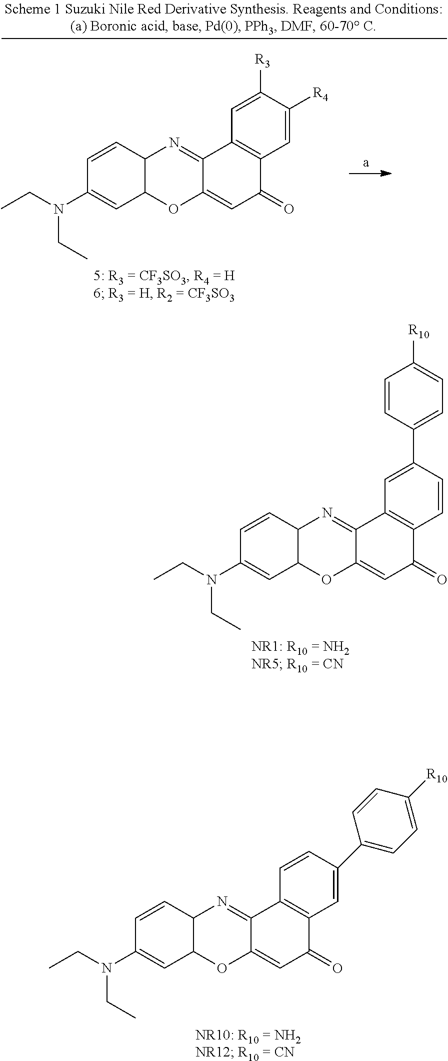

### Photophysical Properties of Nile Red Derivatives

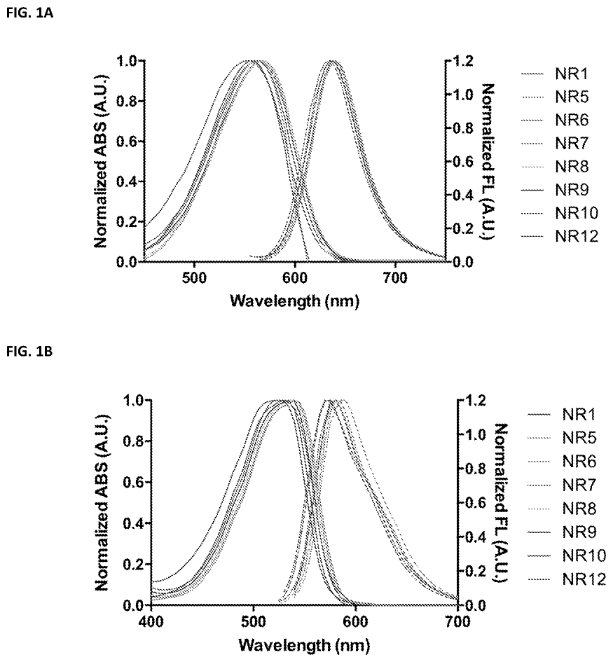

The resulting eight Nile Red derivatives all exhibited similar absorption and emission characteristics to one another as well as to Nile Red (λ_{max ABS}=550 nm, Δ_{max EM}=630 nm in DMSO). Spectral data in DMSO displayed absorbance maxima from all eight Nile Red derivatives between 555-570 nm, while the emission maxima ranged from 635-640 nm (FIG. 1A). Nile Red has well characterized solvochromatic behavior due to a twisted intramolecular charge transfer state formed by the rotation of the diethyl amino group in the excited state. Rotation about this bond only occurs in polar solvents where it is stabilized, resulting in a bathochromic shift.^{23, 33} As expected, all synthesized Nile Red derivatives demonstrated similar solvochromatic behavior to Nile Red (λ_{max ABS}=520 nm, λ_{max EM}=570 nm in toluene) when spectrally evaluated in toluene, with absorbance maxima between 520-545 nm and emission maxima ranging from 575-590 nm (FIG. 1B). The quantum yield was determined for all synthesized derivatives in DMSO and varied widely across the Nile Red derivatives ranging from 0.04-0.56 (Table 1). Notably, derivatives with an electron withdrawing group (NR5, NR6, NR8, NR9 and NR12) were significantly brighter than those with electron donating groups (NR1, NR7 and NR12), where derivatives with electron withdrawing groups had quantum yields >0.45, while derivatives with electron donating groups had quantum yields <0.14.

FIG. 1 depicts Nile Red Derivative absorbance and emission spectra. The normalized absorbance (solid lines) and emission (dashed lines) of NR1, NR5, NR6, NR7, NR8, NR9, NR10 and NR12 are shown in Graph A for Dimethyl Sulfoxide (DMSO) and Graph B for Toluene.

### Nile Red Derivative Cell Staining and Imaging Studies

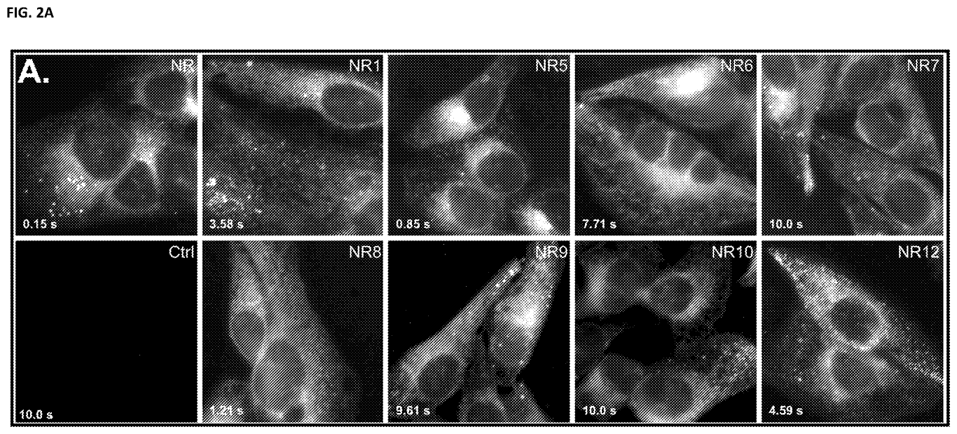

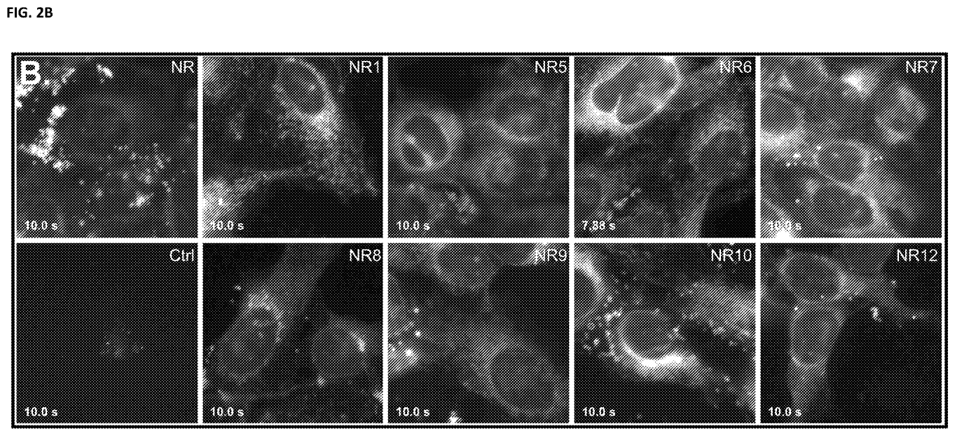

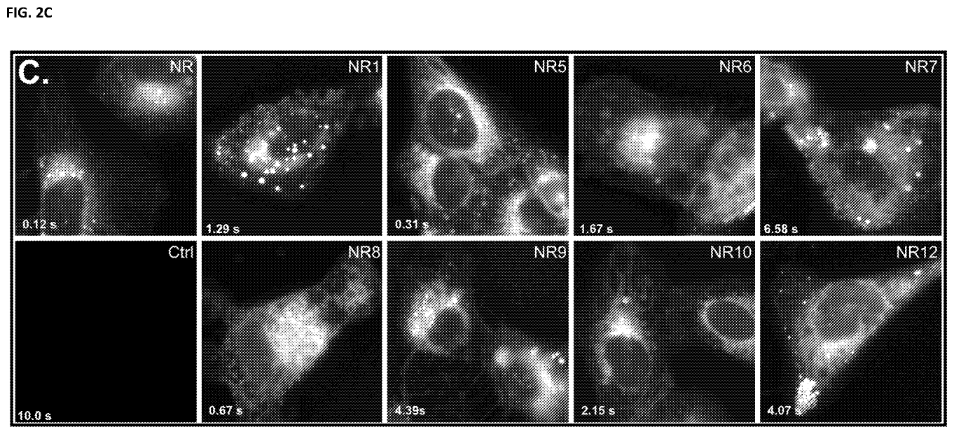

To assess the effect of the Nile Red derivative structural diversity on in vitro labeling, fixed and live cells were stained with Nile Red and the synthesized derivatives (FIG. 2). In fixed cells, the fluorescence intensity was bright for Nile Red as well as NR5 and NR8, where both derivatives contained electron withdrawing groups and had high quantum yields. Interestingly, quantum yield was not directly correlated to in vitro fluorescence intensity since NR1 had a relatively low quantum yield (QY=0.08), but showed brighter in vitro fluorescence than NR12 (QY=0.56) (FIG. 2A). Lipid droplets were the prominently visible in vitro structure with Nile Red as well as its derivatives.

FIG. 2 demonstrates in vitro Nile Red and derivative staining patterns. Nile Red and its derivatives were used to stain U2OS cells that were A) fixed and imaged without washing, B) live and then fixed and washed prior to imaging, and C) live and imaged without washing. Each image was collected at the optimal exposure time and is displayed at ideal contrast to show the visible highlighted intracellular structures using Nile Red and each of its synthesized derivatives.

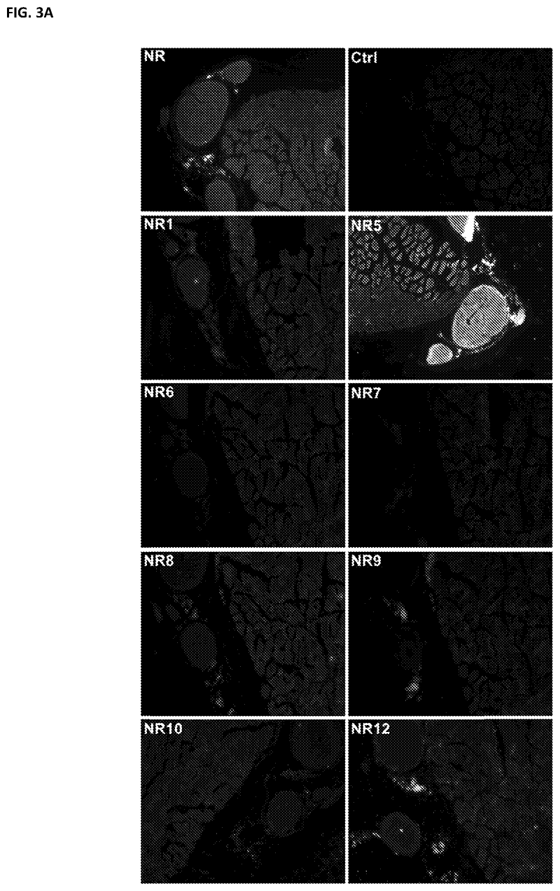

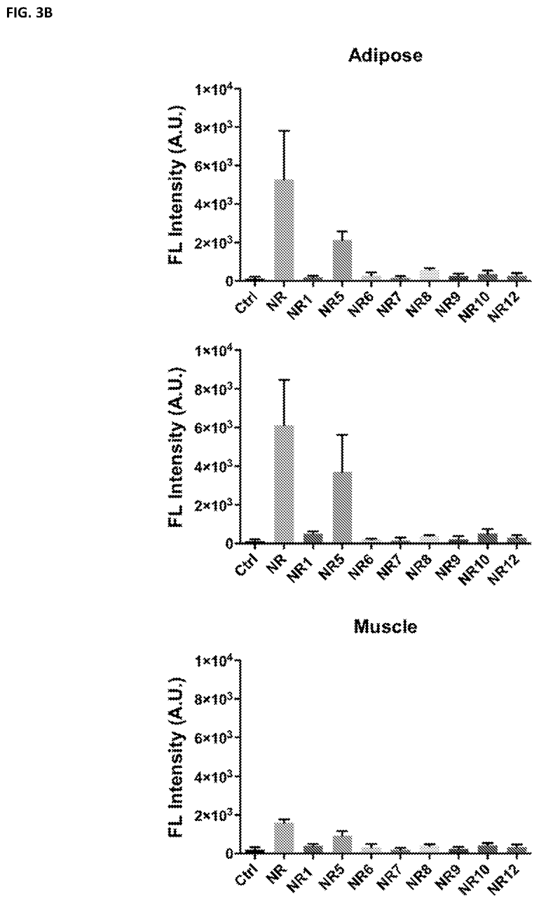

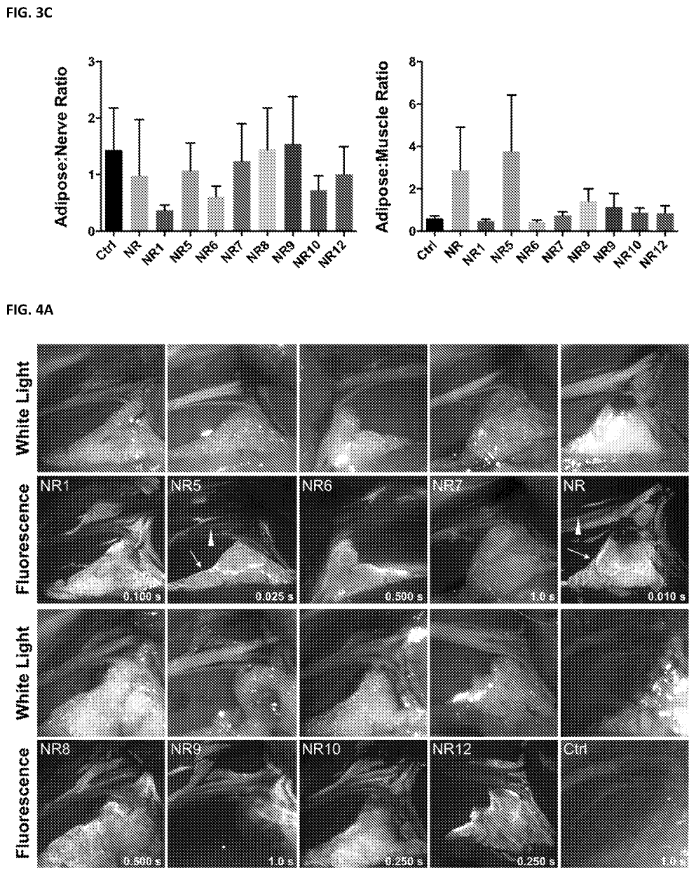

FIG. 3 demonstrates ex vivo Nile Red and derivative staining patterns. A) Nile Red and its synthesized derivatives were used to stain murine tissue sections containing nerve, muscle and adipose tissues. Image were collected at 650 ms and representative images are displayed with equivalent normalization. B) The fluorescence intensity of each tissue type was quantified in n=4 tissue sections per fluorophore, where values were used to compute the C) adipose to nerve and adipose to muscle ratios.

Fluorescence staining patterns of Nile Red and the synthesized derivatives were also assessed in cells that were stained live, then fixed (FIG. 2B). Fluorescence intensity was substantially decreased, as determined from the long exposure times, which was likely due to the extra washing steps used during staining. NR6 was the only derivative to have an exposure time less than 10 sec, suggesting it bound more strongly to subcellular structures than Nile Red or the other Nile Red derivatives. Overall, fluorescent staining of lipid droplet like structures appeared diminished and more cytoplasmic structures were stained when washing was completed prior to imaging. This was particularly evident for NR6, NR7 and NR10.

Finally, the fluorescence staining patterns of Nile Red and the synthesized derivatives were assessed in live cells without washing (FIG. 2C). Fluorescence intensity was generally brightest in this staining study compared to staining of fixed or live cells that were fixed prior to imaging, as determined by the short exposure times. This increase in fluorescence signal was likely due to the lack of washing in the staining procedure. Nile Red was again the brightest overall, with NR5 and NR8 being the brightest of the synthesized derivatives. Prominent lipid droplet like structures were observed for Nile Red, NR1, NR5, NR9 and NR12. Despite the low quantum yield of NR1, it was again brighter than other Nile Red derivatives with higher quantum yields such as NR12 and NR6, as determined based on optimal exposure time used for image collection.

### Nile Red Derivative Ex Vivo Tissue Staining

A subclass of oxazine derivatives with structural similarity to Nile Red and the synthesized derivatives have demonstrated significant nerve specificity.²⁶ To determine if any of the structural changes to the Nile Red scaffold in the synthesized derivatives affected the staining specificity for adipose, muscle or nerve, the synthesized derivatives were used to stain mouse tissue sections containing mouse sciatic nerve as well as the surrounding muscle and adipose tissues (FIG. 3). Nile Red showed adipose specificity as did NR5 and NR12, while little fluorescence staining was seen from the other Nile Red derivatives on mouse tissue sections (FIG. 3A). Region of interest fluorescence intensity analysis showed strong nerve and adipose fluorescence staining from Nile Red and NR5, with substantially lower muscle fluorescence staining intensity (FIG. 3B). The adipose to nerve ratio for Nile Red and all of its derivatives was similar to autofluorescence adipose to nerve ratio, showing minimal contrast increase from fluorophore staining. Notably, adipose to muscle ratio showed increased contrast over control autofluorescence adipose to muscle ratio for Nile Red and NR5 (FIG. 3C).

### Nile Red Derivative In Vivo Tissue Staining & Ratiometric Imaging

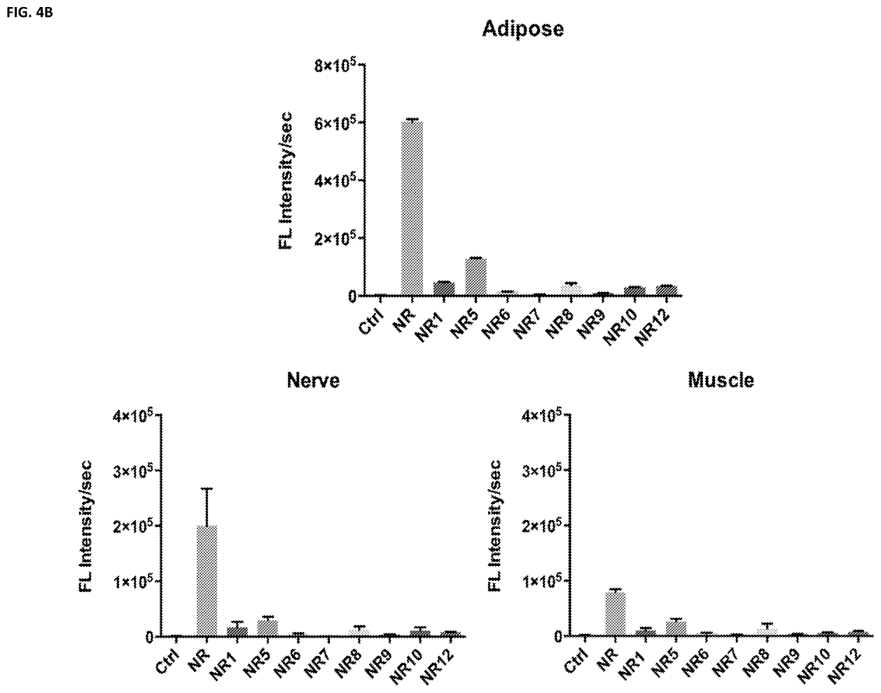

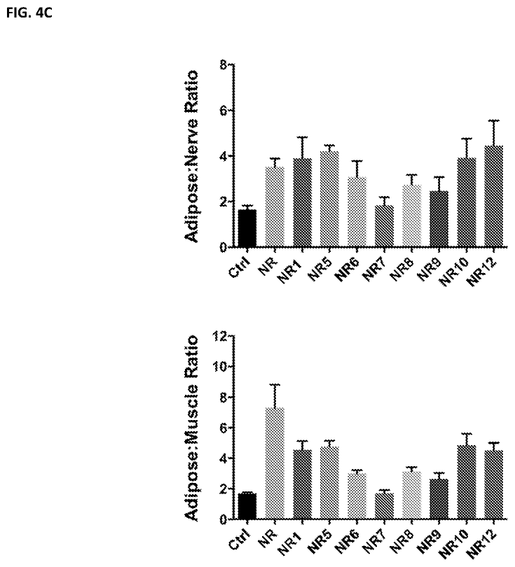

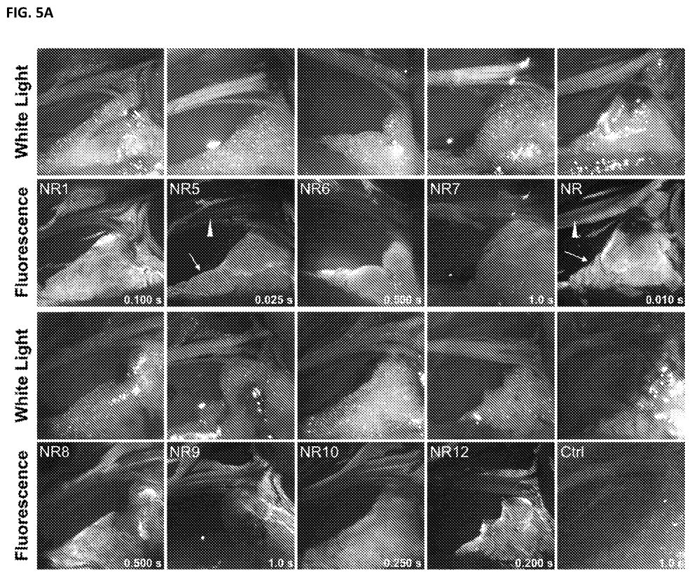

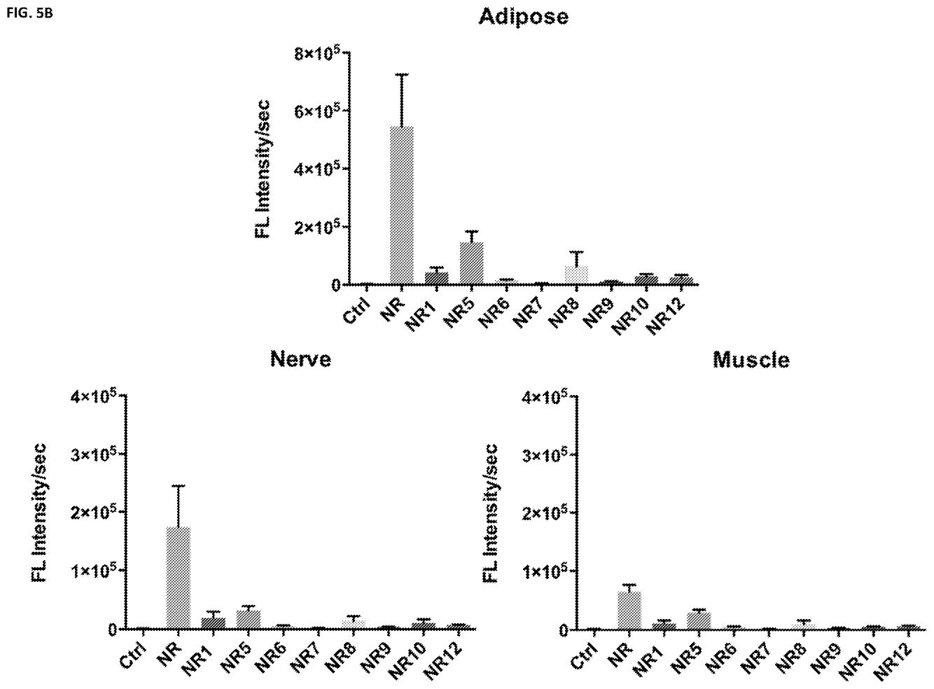

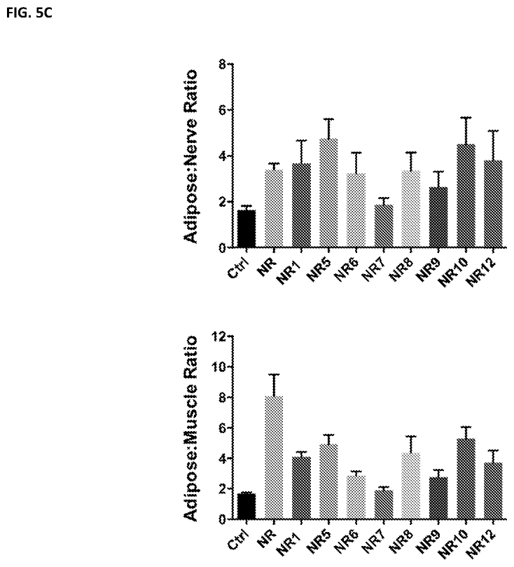

To further assess the tissue specificity of the synthesized Nile Red derivatives, in vivo studies were completed where formulated Nile Red and its derivatives were applied to the sciatic nerves and brachial plexus nerves using an optimized direct administration protocol,² which facilitated similar application to the ex vivo frozen tissue section staining. Fluorescence imaging was completed immediately following staining (FIG. 4) and 30 minutes after completion of staining (FIG. 5) to determine the utility for contrast during a surgical procedure. In vivo imaging immediately following direct administration showed Nile Red and its derivatives localized to the adipose tissue (FIG. 4A). Tissue specific fluorescence analysis showed the greatest fluorescence intensity in the adipose tissue, followed by the nerve then muscle tissues. Nile Red and NR5 had the highest fluorescence signal compared to the other Nile Red derivatives, with Nile Red substantially higher than NR5 (FIG. 4B). Adipose to nerve ratios showed increased contrast over control autofluorescence with similar adipose to nerve contrast from Nile Red, NR1, NR5, NR6, NR10 and NR12. Adipose to muscle ratios also showed greater contrast compared to control autofluorescence with the greatest adipose to muscle contrast seen in the Nile Red stained tissues. While lower than Nile Red, adipose to muscle contrast was similar for NR1, NR5, NR10 and NR12 (FIG. 4C).

FIG. 4 depicts in vivo tissue specific Nile Red & derivative staining immediately after direct administration. A. Nile Red and its synthesized derivatives were used for in vivo direct administration on the brachial plexus and sciatic nerves of mice for n=4 nerve sites/fluorophore. Representative images of the sciatic nerve shown in the white light illuminated surgical field of view and their corresponding fluorescence images are shown, where images were collected immediately after completion of staining at the ideal exposure time per fluorophore. Each image is displayed at its ideal contrast. The adipose tissue for NR5 and Nile Red is depicted by the arrow, while the nerve tissue is depicted by the arrowhead. B. The fluorescence intensity of adipose, muscle and nerve tissue was quantified for each of the n=4 nerve sites/fluorophore, where values were used to compute the C. adipose to nerve and adipose to muscle ratios.

Fluorescence imaging 30 minutes after direct administration showed qualitatively similar nerve, adipose and muscle staining patterns to those observed immediately following direct administration (FIGS. 4A and 5A). Tissue specific fluorescence analysis showed a decrease in adipose, nerve and muscle fluorescence for Nile Red compared to imaging immediately following direct administration. However, NR5 showed slightly increased adipose-specific fluorescence intensity, while nerve and muscle tissue fluorescence were unchanged (FIGS. 4B and 5B). Adipose to nerve contrast was higher than control autofluorescence for Nile Red and all its derivatives except NR7 30 mins post direct administration. Notably, NR5 had higher adipose-to-nerve contrast compared to Nile Red 30 minutes post direct administration, while adipose to muscle contrast was greatest for Nile Red (FIG. 5C). NR5 tissue-specific contrast was also assessed out to 120 minutes after direct administration, where adipose signal was maintained throughout the 120 minute imaging time.

FIG. 5 depicts in vivo tissue specific Nile Red & derivative staining 30 minutes after direct administration. FIG. 5A shows Nile Red and its synthesized derivatives used for in vivo direct administration on the brachial plexus and sciatic nerves of mice for n=4 nerve sites/fluorophore. Representative images of the sciatic nerve shown in the white light illuminated surgical field of view and their corresponding fluorescence images are shown, where images were collected 30 minutes after completion of staining at the ideal exposure time per fluorophore. Each image is displayed at its ideal contrast. The adipose tissue for NR5 and Nile Red is depicted by the arrow, while the nerve tissue is depicted by the arrowhead. FIG. 5B shows the fluorescence intensity of adipose, muscle and nerve tissue was quantified for each of the n=4 nerve sites/fluorophore, where values were used to compute the adipose to nerve and adipose to muscle ratios seen in FIG. 5C.

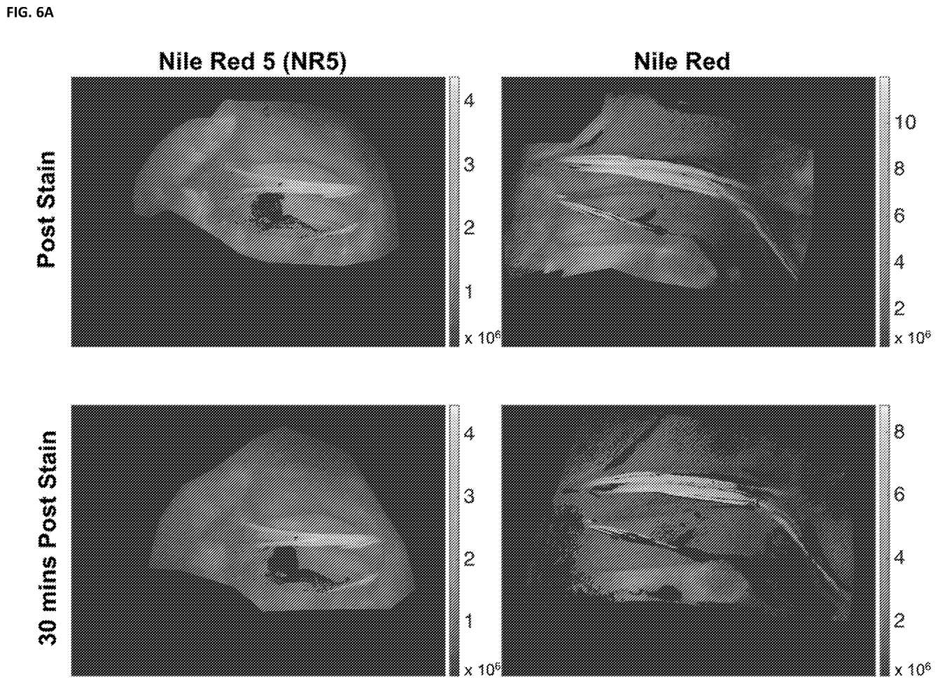

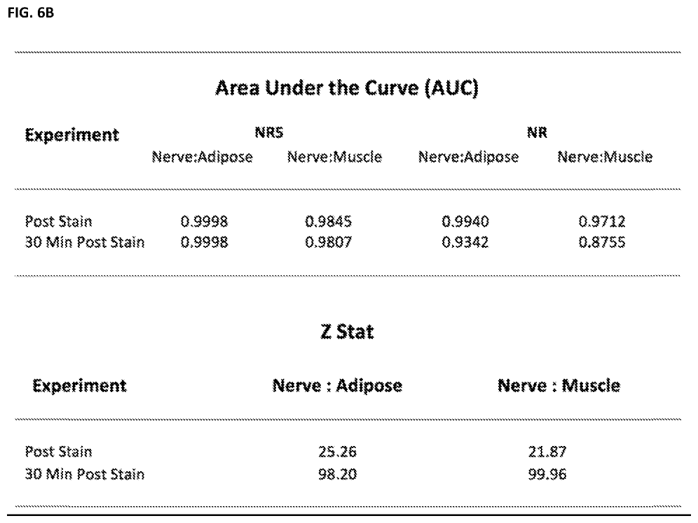

To further assess the in vivo adipose and nerve specificity differences between Nile Red and NR5, ratiometric imaging was completed using Nile Red or NR5 and Oxazine 4 co-staining, both of which demonstrated high nerve contrast (FIG. 6A). ROC curve analysis was completed to quantitatively assess nerve contrast differences between Nile Red and NR5 with Oxazine 4 costaining. Area under the curve (AUC) calculations from the ROC curve analysis showed similar nerve to adipose and nerve to muscle contrast ratios for NR5 and Nile Red immediately following direct administration (FIG. 6B). However, the ROC AUC analysis showed improved nerve to adipose and nerve to muscle contrast ratios for NR5 compared to Nile Red 30 minutes post direct administration demonstrating the utility of NR5 for improved nerve contrast in the surgical setting where adipose and nerve tissue are in close proximity (FIG. 6B).

Although all AUC values were >0.95, the differences in nerve-to-adipose contrast using ratiometric imaging compared to oxazine 4 staining alone were found to be statistically significant (z-score % 25.26 for NR versus NR5 costaining with oxazine 4, z-score % 28.63 for oxazine 4 only staining versus NR5 costaining with oxazine 4). ROC AUC analysis demonstrated that NR5 and oxazine 4 costaining provided significantly higher nerve-to-adipose contrast than Nile Red and oxazine 4 costaining or oxazine 4 staining alone 30 min after staining was completed (z-score % 98.20 for NR versus NR5 costaining with oxazine 4, z-score % 16.12 for oxazine 4 only staining versus NR5 costaining with oxazine 4). ROC AUC analysis of nerve-to-muscle contrast immediately following staining showed slightly improved values for NR5 costaining with oxazine 4 compared to Nile Red and oxazine 4 costaining or oxazine 4 only staining. Calculation of the zscore revealed that the difference in AUC for NR5 and oxazine 4 costaining versus either Nile Red and oxazine 4 costaining or oxazine 4 alone were statistically significant (z-score % 21.87 for NR versus NR5 costaining with oxazine 4, z-score % 15.22 for oxazine 4 only staining versus NR5 costaining with oxazine 4). ROCAUC analysis of the nerve-to-muscle contrast 30 min after staining revealed little change for NR5 and oxazine 4 costaining and improved values for oxazine 4 staining alone, while Nile Red and oxazine 4 costaining show a substantial drop in AUC value. Calculation of the z-score showed that NR5 costaining with oxazine 4 had significantly better nerve to muscle contrast compared to Nile Red costaining with oxazine 4 (z-score % 99.96). However, nerve-to-muscle contrast was not significantly better 30 min after staining with NR5 and oxazine 4 costaining versus oxazine 4 alone (z-score %-21.32).

FIG. 6 depicts in vivo ratiometric tissue specific staining after direct administration. Figure A shows ratiometric images calculated following in vivo co-staining with NR5 and Oxazine 4 (left) or Nile Red and Oxazine 4 (right). FIG. 6 B lists Receiver Operator Characteristic (ROC) curve analysis was performed to assess differences between the ratiometric images, where area under the curve (AUC) was used as the metric to differentiate between the NR5/Oxazine 4 and Nile Red/Oxazine 4 co-stained tissues.

### Discussion

Current surgical techniques rely heavily on a surgeons' ability to identify normal and diseased tissues using visual and tactile cues during surgery, where outcome is strongly influenced by a surgeon's experience and skill level. The peripheral nervous system is responsible for voluntary and involuntary activities and critically important to bodily physical functions and maintenance of biochemical homeostasis. Thus, surgical peripheral nerve damage can be devastating with potentially lifelong, deleterious ramification for affected patients' quality of life. Fluorescence guided surgery provides an opportunity for rapid, high contrast identification of specific tissue types, such as nerves. However, there are currently few clinically approved fluorophores, none of which are nerve specific. The oxazine fluorophore scaffold presents a unique chemical scaffold for tissue-specific fluorescence as modifications to its backbone structure result in both nerve- and adipose-specific fluorescent contrast. Accurate intraoperative nerve identification using nerve-specific fluorophores is often hampered by surrounding adipose tissue, since the molecular composition of nerve and adipose tissues are similar. Therefore, small molecule fluorophores that distribute and accumulate in nerve tissue often demonstrate some degree of off target adipose accumulation and contrast. To improve nerve-specific fluorescent contrast for intraoperative guidance, a library of oxazine-based, adipose-specific fluorophores was designed and synthesized to enable ratiometric imaging for enhancement of nerve-specific intraoperative contrast when used as a costain with nerve-specific fluorophores that demonstrate mixed nerve and adipose accumulations.

The Nile Red scaffold was chosen as the backbone fluorophore for library synthesis due to its known lipophilicity and adipose highlighting properties. While Nile Red itself does provide adipose-specific fluorescence, it also stains nerve tissue, diminishing nerve to adipose tissue contrast by ratiometric imaging (FIGS. 4A, 5A and 6A). The Nile Red scaffold was modified (Schemes 1-3) to diminish nerve specificity while maintaining the adipose specificity for improved nerve contrast using a ratiometric imaging approach. The introduction of a variety of chemical moieties was completed through a conventional nitroso condensation with dihydroxynapthalene to yield a 2- or 3-hydroxy Nile Red derivative. By converting the hydroxyl moiety to a reactive triflate, the core structure was further functionalized using either Suzuki or Sonogashira cross coupling reactions^{30, 32} resulting in 8 novel Nile Red derivatives (NR1, NR5, NR6, NR7, NR8, NR9, NR10 and NR12) (Schemes 2-3).

The Nile Red derivatives were initially screened using in vitro staining to examine the lipid specificity and any solvochromatic behavior. Nile Red is often utilized to stain lipid-like structures in vitro and the synthesized Nile Red derivatives showed similar staining patterns to the parent Nile Red fluorophore (FIG. 2). In vitro staining studies were completed using live and fixed cells, where the only staining differences were seen when washing steps were used prior to imaging, which were not required due to the fluorogenic nature of Nile Red and its derivatives. In both the fixed and live in vitro imaging studies, NR5 demonstrated the brightest fluorescence intensity of the Nile Red derivatives (FIG. 2). Interestingly, even though the quantum yield varied across the derivatives (Table 1), it was not necessarily correlated to the brightness of in vitro staining as demonstrated by comparison of images from NR1 (QY=0.08) and NR12 (QY=0.56) (FIGS. 2A and 2C).

TABLE 1
Optical Properties of Nile Red Derivatives in DMSO.

| NR | Max | Max | Stokes | |
|---|---|---|---|---|
| Derivative | Abs (nm) | Em (nm) | Shift (nm) | QY |
| NR1 | 555 | 635 | 80 | 0.08 |
| NR5 | 560 | 640 | 80 | 0.54 |
| NR6 | 570 | 640 | 70 | 0.45 |
| NR7 | 565 | 640 | 75 | 0.14 |
| NR8 | 565 | 640 | 75 | 0.48 |
| NR9 | 565 | 640 | 75 | 0.48 |
| NR10 | 555 | 635 | 80 | 0.04 |
| NR12 | 560 | 635 | 75 | 0.56 |

To assess their utility for enhanced nerve-specific fluorescent contrast, the Nile Red derivatives were screened for tissue specificity ex vivo on rodent sciatic nerve sections that contained nerve, adipose and muscle tissues. NR5 demonstrated the brightest overall tissue staining compared to the other Nile Red derivatives (FIG. 3A). Fluorescence intensity quantification per tissue showed a substantially higher adipose, nerve and muscle tissue fluorescence for NR5 compared to the other derivatives, however the parent Nile Red fluorophore demonstrated the highest overall tissue fluorescence (FIG. 3B). Adipose to nerve and adipose to muscle tissue contrast ratios were also quantified, where less difference was seen between NR5, the other derivatives or Nile Red. However, adipose to muscle tissue contrast again showed that both Nile Red and NR5 demonstrated the highest contrast ratios (FIG. 3C).

The utility of the novel Nile Red derivatives for adipose-specific signal was also assessed following direct administration in vivo immediately after as well as 30 minutes following the completion of staining (FIGS. 4A and 5A). In vivo direct administration in rodent sciatic and brachial plexus nerves showed strong fluorescence intensity following Nile Red staining in the adipose, nerve and muscle tissues immediately following staining (FIG. 4B). Nile Red staining per tissue decreased over the 30 minute period after staining, while NR5 showed increased adipose intensity with unchanged nerve and muscle tissue fluorescence 30 minutes post direct administration (FIG. 5B). NR5 showed the highest fluorescence intensity in the adipose, nerve and muscle tissues of the synthesized Nile Red derivatives both immediately post stain as well as at 30 minutes after staining (FIGS. 4B and 5B). The adipose to nerve and adipose to muscle ratios were calculated to further assess tissue specific contrast. The mean adipose to nerve ratios were similar for NR1, NR5, NR10, NR12 and the parent Nile Red compound immediately after staining (FIG. 4C). Adiopse to nerve ratio decreased 30 minutes after staining for Nile Red, while it increased for NR5 demonstrating the potential utility of this agent to highlight adipose tissue specificity in the context of nerves (FIGS. 4C and 5C). The change in the adipose to nerve ratio was largely due to an overall decrease in nerve and adipose fluorescence signal in the Nile Red group and an increase in the adipose signal for the NR5 group improving overall contrast (FIGS. 4B and 5B). Adipose to muscle signal was greatest for Nile Red compared to all Nile Red derivatives immediately post stain as well as up to 30 minutes following staining (FIGS. 4C and 5C).

Direct administration using Nile Red and Oxazine 4 co-staining was previously optimized, demonstrating adipose and nerve-specific staining respectively, however Nile Red accumulation in the nerve tissue was visible hindering ratiometric analysis for improved nerve contrast.² NR5 was selected for ratiometric imaging studies due to its tissue-specific staining and adipose to nerve contrast to determine if contrast was improved over Nile Red and Oxazine 4 co-staining. Ratiometric images using both Nile Red and NR5 to specifically highlight the adipose tissue resulted in high contrast immediately post stain as well as 30 minutes after staining (FIG. 6A). ROC curve analysis demonstrated similar nerve contrast when either Nile Red or NR5 were used for adipose-specific staining. Excitingly, ROC AUC demonstrated improvement in nerve contrast 30 minutes post direct administration for NR5 as compared to Nile Red (FIG. 6B).

In summary, a novel Nile Red derivative, NR5 was synthesized and characterized for its adipose specificity in vitro, ex vivo and in vivo. NR5 showed both adipose specific contrast as well as minimal nerve accumulation, differentiating it from the parent compound Nile Red, which demonstrates some nerve accumulation. In vivo co-staining with nerve-specific Oxazine 4 and NR5 showed improved nerve to adipose and nerve to muscle tissue contrast as compared to the parent Nile Red compound due to this decreased nerve accumulation. This contrast improvement was most apparent 30 minutes post direct administration, demonstrating the potential utility for surgical applications. This novel Nile Red derivative could be used in future fluorescence image guided surgery applications where adipose specific contrast or nerve specific contrast were required.

### REFERENCES

1. Macrae, W. A. Chronic post-surgical pain: 10 years on. Br J Anaesth 101, 77-86 (2008).
2. Barth, C. W. & Gibbs, S. L. Direct Administration of Nerve-Specific Contrast to Improve Nerve Sparing Radical Prostatectomy. Theranostics 7, 573-593 (2017).
3. Keereweer, S. et al. Optical image-guided surgery—where do we stand? Molecular imaging and biology: MIB: the official publication of the Academy of Molecular Imaging 13, 199-207 (2011).
4. AV, D. S., Lin, H., Henderson, E. R., Samkoe, K. S. & Pogue, B. W. Review of fluorescence guided surgery systems: identification of key performance capabilities beyond indocyanine green imaging. J Biomed Opt 21, 80901 (2016).
5. Acerbi, F. et al. Fluorescein-guided surgery for malignant gliomas: a review. Neurosurg Rev 37, 547-557 (2014).
6. Eljamel, M. S. & Mahboob, S. O. The effectiveness and cost-effectiveness of intraoperative imaging in high-grade glioma resection; a comparative review of intraoperative ALA, fluorescein, ultrasound and MRI. Photodiagnosis Photodyn Ther 16, 35-43 (2016).
7. Gibbs, S. L. Near infrared fluorescence for image-guided surgery. Quantitative imaging in medicine and surgery 2, 177-187 (2012).
8. Gioux, S., Choi, H. S. & Frangioni, J. V. Image-guided surgery using invisible near-infrared light: fundamentals of clinical translation. Molecular imaging 9, 237-255 (2010).
9. Vahrmeijer, A. L., Hutteman, M., van der Vorst, J. R., van de Velde, C. J. & Frangioni, J. V. Image-guided cancer surgery using near-infrared fluorescence. Nature reviews. Clinical oncology 10, 507-518 (23 Jul. 2013).
10. Wu, C. et al. Molecular probes for imaging myelinated white matter in CNS. Journal of medicinal chemistry 51, 6682-6688 (2008).
11. Wang, C. et al. In situ fluorescence imaging of myelination. The journal of histochemistry and cytochemistry: official journal of the Histochemistry Society 58, 611-621 (2010).
12. Cotero, V. E. et al. Intraoperative fluorescence imaging of peripheral and central nerves through a myelin-selective contrast agent. Molecular imaging and biology: MIB: the official publication of the Academy of Molecular Imaging 14, 708-717 (2012).
13. Gibbs, S. L. et al. Structure-activity relationship of nerve-highlighting fluorophores. PloS one 8, e73493(2013).
14. Gibbs-Strauss, S. L. et al. Nerve-highlighting fluorescent contrast agents for image-guided surgery. Molecular imaging 10, 91-101 (2011).
15. Stankoff, B. et al. Imaging of CNS myelin by positron-emission tomography. Proceedings of the National Academy of Sciences of the United States of America 103, 9304-9309 (2006).
16. Meyers, J. R. et al. Lighting up the senses: FM1-43 loading of sensory cells through nonselective ion channels. The Journal of neuroscience: the official journal of the Society for Neuroscience 23, 4054-4065 (2003).
17. Gibbs-Strauss, S. L. et al. Molecular imaging agents specific for the annulus fibrosus of the intervertebral disk. Molecular imaging 9, 128-140 (2010).
18. Park, M. H. et al. Prototype nerve-specific near-infrared fluorophores. Theranostics 4, 823-833 (2014).
19. Wang, C. et al. Longitudinal near-infrared imaging of myelination. The Journal of neuroscience: the official journal of the Society for Neuroscience 31, 2382-2390 (2011).
20. Fagerholm, U. The highly permeable blood-brain barrier: an evaluation of current opinions about brain uptake capacity. Drug Discov Today 12, 1076-1082 (2007).
21. Waterhouse, R. N. Determination of lipophilicity and its use as a predictor of blood-brain barrier penetration of molecular imaging agents. Molecular imaging and biology: MIB: the official publication of the Academy of Molecular Imaging 5, 376-389 (2003).
22. Greenspan, P., Mayer, E. P. & Fowler, S. D. Nile red: a selective fluorescent stain for intracellular lipid droplets. J Cell Biol 100, 965-973 (1985).
23. Martinez, V. & Henary, M. Nile Red and Nile Blue: Applications and Syntheses of Structural Analogues. Chemistry 22, 13764-13782 (2016).
24. Wei, R. et al. Nile Red Derivative-Modified Nanostructure for Upconversion Luminescence Sensing and Intracellular Detection of Fe(3+) and MR Imaging. ACS Appl Mater Interfaces 8, 400-410(2016).
25. Velapoldi, R. A. & Tonnesen, H. H. Corrected emission spectra and quantum yields for a series of fluorescent compounds in the visible spectral region. J Fluoresc 14, 465-472 (2004).
26. Connor W. Barth, S. L. G. in SPIE BiOS, Vol. 9696 {96960R} (SPIE, 2016).
27. Hackman, K. M. et al. Polymeric Micelles as Carriers for Nerve-Highlighting Fluorescent Probe Delivery. Molecular pharmaceutics (2015).
28. Barth, C. W., Schaefer, J. M., Rossi, V. M., Davis, S. C. & Gibbs, S. L. Optimizing fresh specimen staining for rapid identification of tumor biomarkers during surgery. Theranostics 7, 4722-4734 (2017).
29. S. J. Briggs, M. et al. Synthesis of functionalised fluorescent dyes and their coupling to amines and amino acids. Journal of the Chemical Society, Perkin Transactions 1, 1051-1058 (1997).
30. Mio, M. J. et al. One-Pot Synthesis of Symmetrical and Unsymmetrical Bisarylethynes by a Modification of the Sonogashira Coupling Reaction. Organic Letters 4, 3199-3202 (2002).
31. Miyaura, N. & Suzuki, A. Palladium-Catalyzed Cross-Coupling Reactions of Organoboron Compounds. Chemical Reviews 95, 2457-2483 (1995).
32. Littke, A. F., Dai, C. & Fu, G. C. Versatile Catalysts for the Suzuki Cross-Coupling of Arylboronic Acids with Aryl and Vinyl Halides and Triflates under Mild Conditions. Journal of the American Chemical Society 122, 4020-4028 (2000).
33. Reichardt, C. Solvatochromic Dyes as Solvent Polarity Indicators. Chemical Reviews 94, 2319-2358(1994).

What is claimed:

1. A compound of Formula (I): 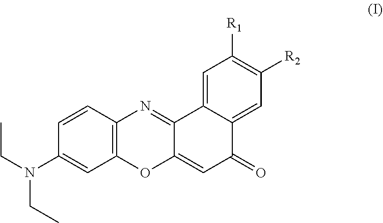
wherein:
one of R₁ and R₂ is hydrogen;
the other of R₁ and R₂ is selected from the group of: 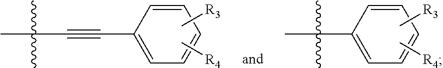
R₃ and R₄ are independently selected from the group of H, —CN, —O—C₁-C₆ alkyl, —NH₂, NH(C₁—C alkyl), —N(C₁-C₆ alkyl)₂, —CH═O, and —SO₂(C₁-C₆ alkyl);
with the proviso that not more than one of R₃ and R₄ is hydrogen.

2. The compound of claim 1, wherein R₃ and R₄ are independently selected from H, —CN, —O—C₁-C₃ alkyl, —NH₂, NH(C₁-C3₄ alkyl), —N(C₁-C₃ alkyl)₂, —CH═O, and —S₂(C₁-C₃ alkyl); with the proviso that not more than one of R₃ and R₄ is hydrogen.

3. The compound of claim 1, wherein R₃ and R₄ are independently selected from H, —CN, —O—CH₃, —NH₂, —NH—CH₃, —N(CH₃)₂, —CH═O, and —SO₂(CH₃); with the proviso that not more than one of R₃ and R₄ is hydrogen.

4. The compound of claim 1, wherein R₃ and R₄ are independently selected from H, —CN, —O—CH₃, —NH₂, —CH═O, and —SO₂(CH₃); with the proviso that not more than one of R₃ and R₄ is hydrogen.

5. The compound of claim 1, wherein:
one of R₁ and R₂ is hydrogen;
the other of R₁ and R₂ is a group of the formula: 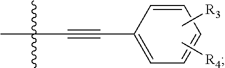
and
R₃ and R₄ are independently selected from H, —CN, —O—C₁-C₆ alkyl, —NH₂, NH(C₁-C₆ alkyl), —N(C₁-C₆ alkyl)₂, —CH═O, and —SO₂(C₁-C₆ alkyl);
with the proviso that not more than one of R₃ and R₄ is hydrogen.

6. The compound of claim 1, wherein:
one of R₁ and R₂ is hydrogen;
the other of R₁ and R₂ is a group of the formula: 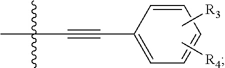
and
R₃ and R₄ are independently selected from H, —CN, —O—C₁-C₃ alkyl, —NH₂, NH(C₁-C₃ alkyl), —N(C₁-C₃alkyl)₂, —CH═O, and —SO₂(C₁-C₃ alkyl);
with the proviso that not more than one of R₃ and R₄ is hydrogen.

7. The compound of claim 1, wherein:
one of R₁ and R₂ is hydrogen;
the other of R₁ and R₂ is a group of the formula: 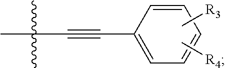
and
R₃ and R₄ are independently selected from H, —CN, —O—CH₃, —NH₂, —NH—CH₃, —N(CH₃)₂, —CH═O, and —SO₂(CH₃);
with the proviso that not more than one of R₃ and R₄ is hydrogen.

8. The compound claim 1, wherein:
one of R₁ and R₂ is hydrogen;
the other of R₁ and R₂ is a group of the formula: 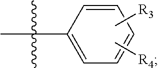
and
R₃ and R₄ are independently selected from H, —CN, —O—C₁-C₆ alkyl, —NH₂, NH(C₁-C₆ alkyl), —N(C₁-C₆ alkyl)₂, —CH═O, and —SO₂(C₁-C₆ alkyl);
with the proviso that not more than one of R₃ and R₄ is hydrogen.

9. The compound of claim 1, wherein:
one of R₁ and R₂ is hydrogen;
the other of R₁ and R₂ is a group of the formula: 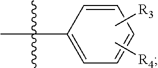
and
R₃ and R₄ are independently selected from H, —CN, —O—C₁-C₃ alkyl, —NH₂, NH(C₁-C₃ alkyl), —N(C₁-C₃alkyl)₂, —CH═O, and —SO₂(C₁-C₃ alkyl);
with the proviso that not more than one of R₃ and R₄ is hydrogen.

10. The compound of claim 1 wherein:
one of R₁ and R₂ is hydrogen;
the other of R₁ and R₂ is a group of the formula: 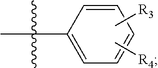
and
R₃ and R₄ are independently selected from H, —CN, —O—CH₃, —NH₂, —NH—CH₃, —N(CH₃)₂, —CH═O, and —SO₂(CH₃);
with the proviso that not more than one of R₃ and R₄ is hydrogen.

11. A compound of claim 1, selected from the group of:
4-((9-(diethylamino)-5-oxo-5H-benzo[a]phenoxazin-2-yl)ethynyl)-2-(methylsulfonyl)benzaldehyde;
2-((3-aminophenyl)ethynyl)-9-(diethylamino)-5H-benzo[a]phenoxazin-5-one;
3-((9-(diethylamino)-5-oxo-5H-benzo[a]phenoxazin-2-yl)ethynyl)benzaldehyde;
3-((9-(diethylamino)-5-oxo-5H-benzo[a]phenoxazin-2-yl)ethynyl)-4-methoxybenzaldehyde;
(3-(4-aminophenyl)-9-(diethylamino)-5H-benzo[a]phenoxazin-5-one;
4-(9-(diethylamino)-5-oxo-5H-benzo[a]phenoxazin-3-yl)benzonitrile;
2-(4-aminophenyl)-9-(diethylamino)-5H-benzo[a]phenoxazin-5-one; and
4-(9-(diethylamino)-5-oxo-5H-benzo[a]phenoxazin-2-yl)benzonitrile.

12. The use of the compound of claim 1 in nerve-specific ratiometric imaging.

13. The use of the compound of claim 1 for fluorescence image guided surgery.
